SEQUENCE LISTING (3 sequences)

SEQ ID NO: 1 - protein, 243 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRICLKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGGGGLEHHHHHH

SEQ ID NO: 2 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 453 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESLTQPEGKNNTTKPPKVSNKALPAPIEKTISKSRKQPREPQVYTLPPSREEMTKNQVSLTCLVKSLKSPK

(19) United States
(12) Patent Application Publication
(10) Pub. No.: US 2026/0337157 A1
(43) Pub. Date: Oct. 29, 2026

(54) CONJUGATE AND THE PREPARATION THEREOF

(71) Applicant: NANJING UNIVERSITY, Jiangsu (CN)

(72) Inventors: Jinbiao JIAO, Jiangsu (CN); Jie LI, Jiangsu (CN); Qingsong WU, Jiangsu (CN); Yudi HU, Jiangsu (CN); Mingxin FAN, Jiangsu (CN)

(73) Assignee: NANJING UNIVERSITY, Jiangsu (CN)

(21) Appl. No.: 19/484,471

(22) PCT Filed: May 8, 2024
(86) PCT No.: PCT/CN2024/091644

§ 371 Date: Nov. 14, 2025

(30) Foreign Application Priority Data
May 16, 2023 (WO) PCT/CN2023/094511

(51) Int. Cl. C07C 309/83; A61K 47/54; A61K 47/65

(52) U.S. Cl. CPC C07C 309/83; A61K 47/545; A61K 47/65

(57) ABSTRACT

Provided herein are a conjugate and the preparing method and use thereof. The conjugate has the structure of M-[L]ₐ (I), wherein, M is a biological macromolecule, which is linked to L with a nucleophilic functional group comprised in M; a is an integer of 1 to 8; L is a compound of formula (Ia), wherein, R ₁ is an electron withdrawing group; R ₂ comprises a functional molecule.

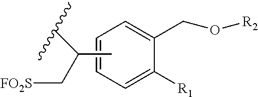

### BACKGROUND OF THE INVENTION

The modification of a protein may give the protein new properties and functions. The common protein modification strategy is to modify the amino acid residues on the protein. Because lysine and cysteine have high affinity reactivity, the modification of the protein often occurs on these two amino acids.

In 2019, three antibody-conjugated drugs Fam-trastuzumab Deruxtecan (for treating HER2-positive unresectable or metastatic breast cancer), Polatuzumab Vedotin (for treating relapsed/refractory diffuse large B cells Lymphoma) and Enfortumab Vedotin (for treating locally advanced or metastatic urothelial cancer) were approved by the FDA, and are achieved by modifying the reduced interchain disulfide bonds on the antibody, and the linkers thereof are maleimide. It is well known that the adducts of maleimide and sulfhydryl are unstable and prone to thiol exchange in physiological condition. The ADC synthesized by the addition reaction between the maleimide and the sulfhydryl may cause the drug to be released in advance, which may seriously affect the stability and efficacy of the ADC.

Although chemists have achieved results in improving the stability of maleimide and seeking new stable linkers, the problems have not been overcome yet. Hence, a kind of linker satisfying the demand of preparing the conjugate, especially in balancing the activity and stability, is urgent to be developed.

### SUMMARY OF THE INVENTION

The present application provides a compound, a conjugate prepared from the compound, and a method preparing the conjugate. The compound of the present application has a high reactivity for preparation the conjugate. The preparation method of the present application may be combined with other methods for preparation a conjugate to produce conjugate with different drugs.

In one aspect, the present application provides a compound of formula (Ib),

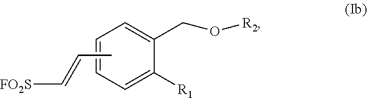

wherein, R₁ is an electron withdrawing group; and R₂ comprises a functional molecule.

In some embodiments, R₁ is selected from —CN, —NO₂, —CHO, fluoro, trifluoro methyl, acetoxy, sulfonic acid, —COR₁₁, —COOR₁₂, wherein, R₁₁ and R₁₂ is independently selected from C₁-C₆ alkyl. In some embodiments, R₁ is —CN. In some embodiments, R₁₁ is C₁-C₃ alkyl. In some embodiments, R₁₂ is C₁-C₃ alkyl.

In some embodiments, the compound is

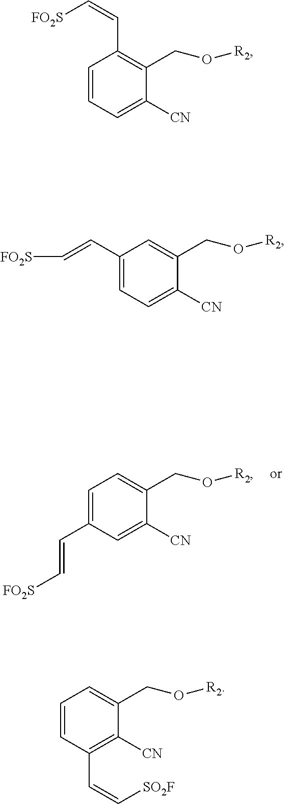

In some embodiments, the functional molecule in R₂ is a chemical reactive group.

In some embodiments, functional molecule in R₂ is selected from azido, alkynyl, cyclohydrocarbyl, alkyne, —NH₂, carboxyl, and tetrazine.

In some embodiments, the functional molecule in R₂ is selected from —COOH, —NH₂, —N₃, ethynyl,

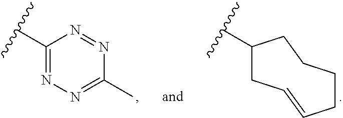

In some embodiments, the compound is selected from the group consisting of:

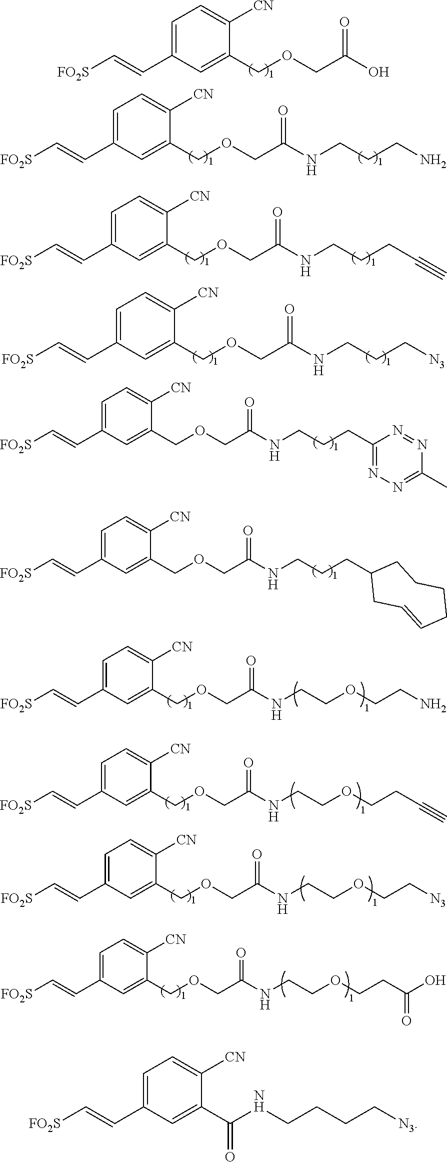

In another aspect, the present application provides a conjugate of formula (I): M-[L]ₐ (I), wherein, M is a biological macromolecule, which is linked to L with a nucleophilic functional group comprised in M; a is an integer of 1 to 8; L is a compound of formula (Ia):

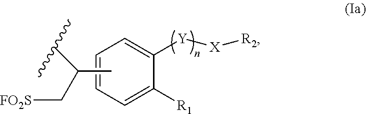

wherein, Y is —C—, an electron withdrawing group or an electron donating group, X is —O—, —N— or —C—; R₁ is an electron withdrawing group; R₂ comprises a functional molecule; n is an integer of 1 to 10.

In some embodiments, Y is —C—. In some embodiments, X is —O—.

In some embodiments, R₁ is selected from —CN, —NO₂, —CHO, fluoro, trifluoro methyl, acetoxy, sulfonic acid, —COR₁₁, —COOR₁₂, wherein, R₁₁ and R₁₂ is independently selected from C₁-C₆ alkyl.

In some embodiments, R₁ is —CN.

In some embodiments, Y is —C—, X is —O—, R₁ is —CN, and n is an integer of 1-3.

In some embodiments, L is

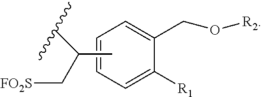

In some embodiments, L is

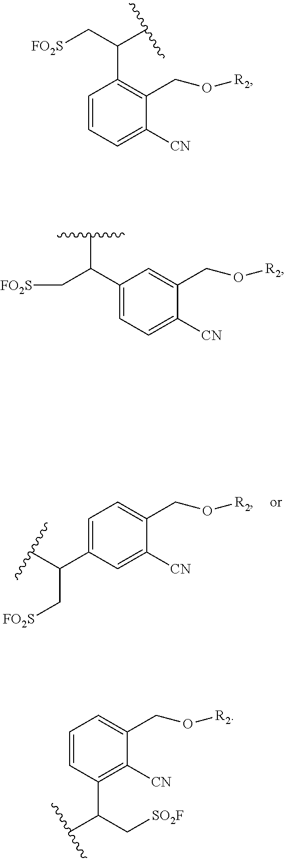

In some embodiments, the nucleophilic functional group of M is selected from a group consisting of —SH, —NH₂, —SeH, —OH, and

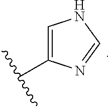 [SMILES: *c1c[nH]cn1]

In some embodiments, M is a protein. In some embodiments, M is an antibody.

In some embodiments, the functional molecule in R₂ is a chemical reactive group.

In some embodiments, the functional molecule in R₂ is selected from azido, alkynyl, cyclohydrocarbyl, alkyne, —NH₂, carboxyl, and tetrazine.

In some embodiments, the functional molecule in R₂ is selected from—COOH, —NH₂, —N₃, ethynyl,

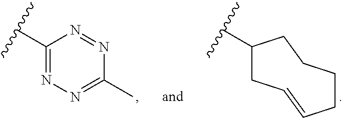

In some embodiments, R₂ comprise a functional molecule selected from a toxin, a fluorescent dye, or a biotin. In some embodiments, R₂ comprise a functional molecule capable of inhibiting the growth of a tumor cell. In some embodiments, R₂ comprise a functional molecule selected from the group consisting of a DNA topoisomerase I inhibitor, a V-ATPase inhibitor, a pro-apoptotic agent, a Bc12 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a nucleoside analog, a HD AC inhibitor; an anthracycline; a NAMPT inhibitor; a hydrophilic prodrug; a SN-38 glucuronide, a etoposide phosphate; a nitrogen mustard, a proteosome inhibitor, a cytokine, a Toll-like receptor agonist and a Sting agonist. In some embodiments, R₂ comprises Dxd. In some embodiments, R₂ comprises doxorubicin, eribulin, and/or MMAE.

In some embodiments, R₂ comprise a linker which is selected from the group consisting of a cleavable linker, a non-cleavable linker, a hydrophilic linker, a hydrophobic linker, a procharged linker, an uncharged linker and a dicarboxylic acid-based linker. In some embodiments, R₂ comprise a linker which is selected from the group consisting of VC-PAB, N-succinimidyl-3-(2-pyridyldithio) propionate (SPDP), N-succinimidyl 4-(2-pyridyldithio) pentanoate (SPP), N-succinimidyl 4-(2-pyridyldithio) butanoate (SPDB), N-succinimidyl-4-(2-pyridyldithio)-2-sulfo-butanoate (sulfo-SPDB), iodoacetate N-succinimidyl (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1). In some embodiments, R₂ comprise a linker and the linker comprising (PEG) n, wherein n is an integer selected from 2-10.

In some embodiments, R₂ is -Linker-FM, wherein FM is the functional molecule.

In some embodiments, a is an integer selected from 1-10. In some embodiments, a is an integer selected from 2-8. In some embodiments, a is an integer selected from 2-4. In some embodiments, a is 2, 4,6 or 8. In some embodiments, a is 2, 4,6 or 8. In some embodiments, the conjugate comprises 2, 4, 6, or 8 constructs of formula (Ia), preferably 2 or 4.

In some embodiments, L is selected from the group consisting of:

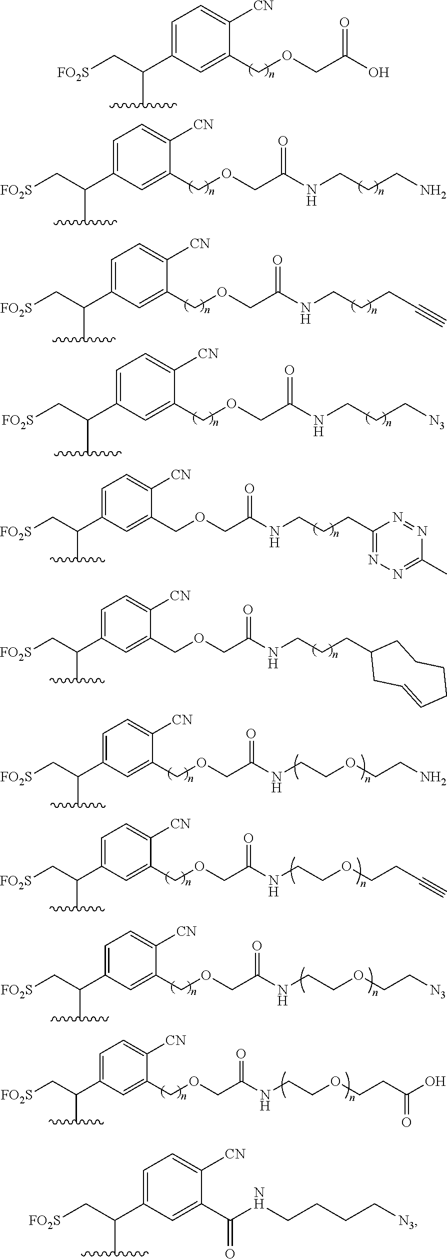

wh erein n is an integer selected from 0-20. For example, n is an integer selected from 1-10. For example, n is an integer selected from 1-8. For example, n is an integer selected from 1-6. For example, n is 1. For example, n is 2. For example, n is 3.

In some embodiments, the conjugate is

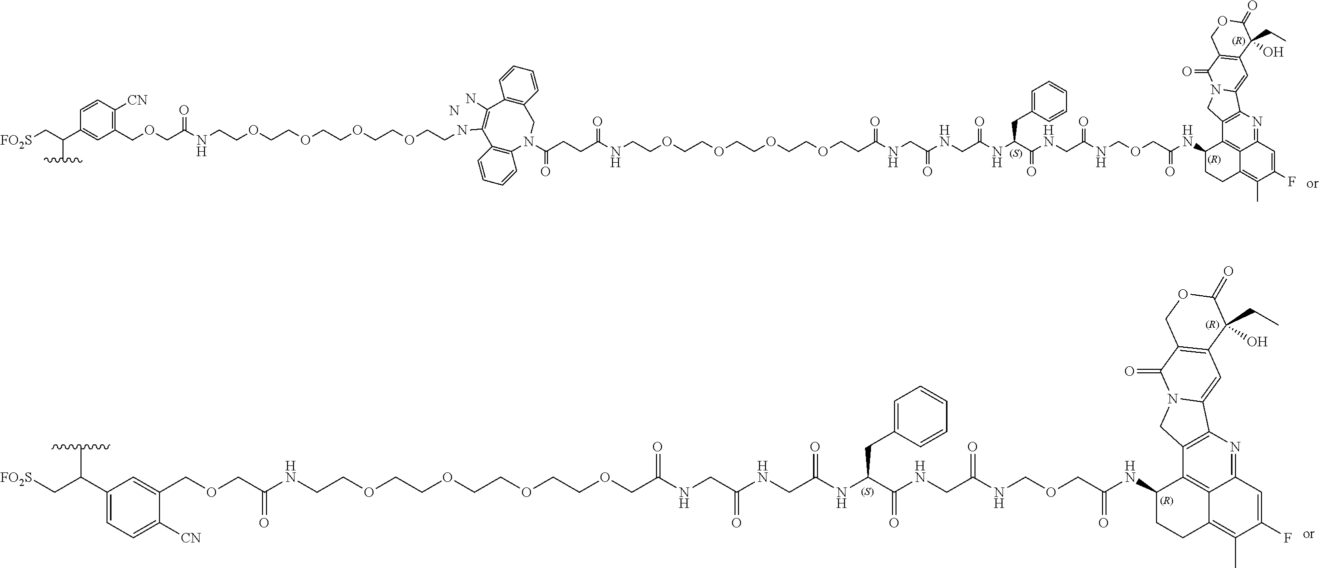

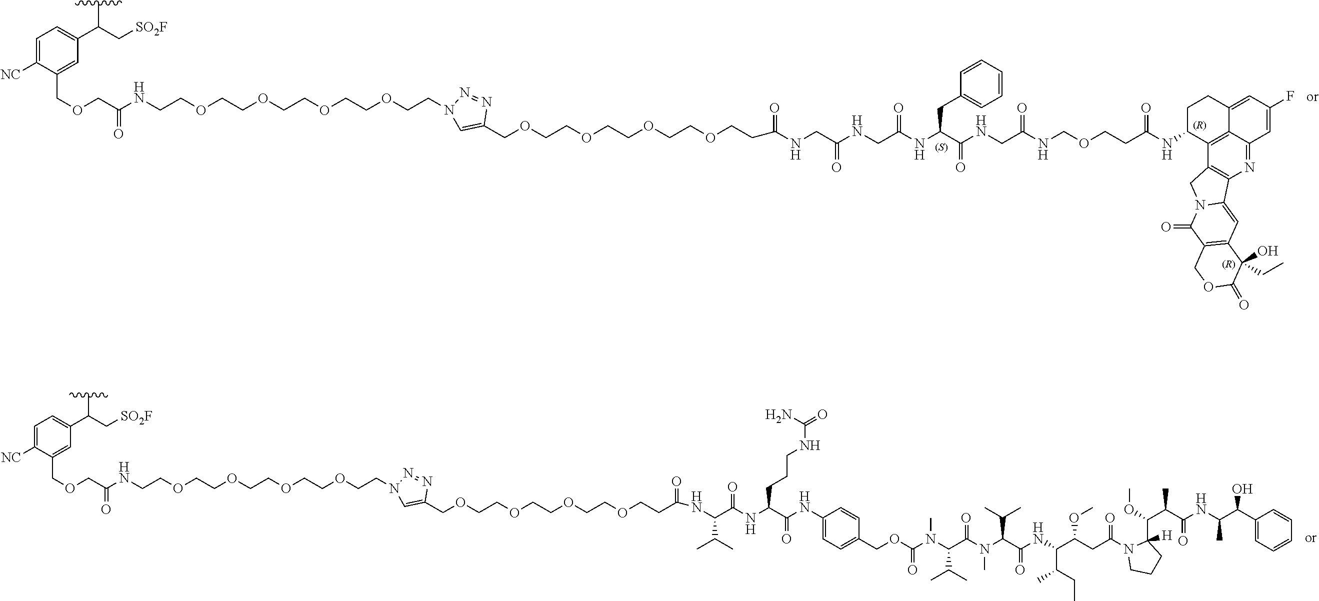

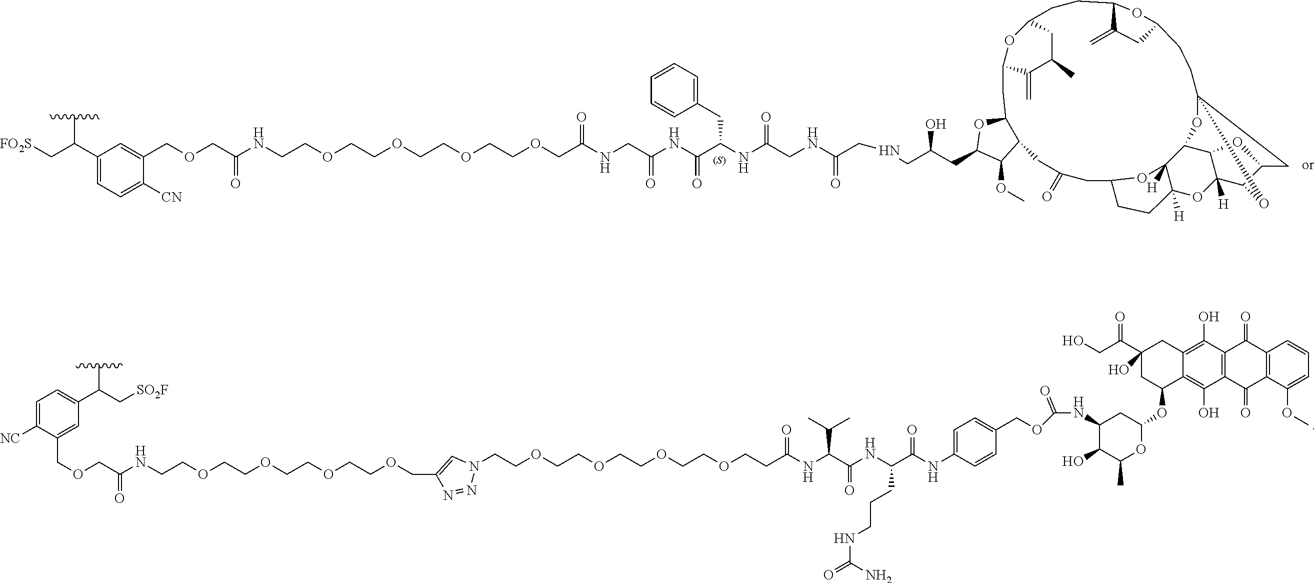

In another aspect, the present application provides a method for preparing the conjugate of the present application.

In another aspect, the present application provides a method for preparation a conjugate, comprising contacting a compound of formula (Ib):

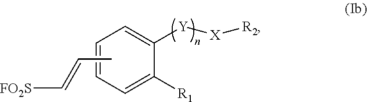

to M, wherein, M is a biological macromolecule, which comprising a nucleophilic functional group; Y is —C—, an electron withdrawing group or an electron donating group, X is —O—, —N— or —C—; R₁ is an electron withdrawing group; R₂ comprises a functional molecule; n is an integer of 1 to 10.

In some embodiments, Y is —C—. In some embodiments, X is —O—.

In some embodiments, R₁ is selected from—CN, —NO₂, —CHO, fluoro, trifluoro methyl, acetoxy, sulfonic acid, —COR₁₁, —COOR₁₂, wherein, R₁₁ and R₁₂ is independently selected from C₁-C₆ alkyl. In some embodiments, R₁₁ is C₁-C₃ alkyl. In some embodiments, R₁₂ is C₁-C₃ alkyl.

In some embodiments, R₁ is —CN.

In some embodiments, Y is —C—, X is —O—, R₁ is —CN, and n is an integer of 1-3.

In some embodiments, formula (Ib) is

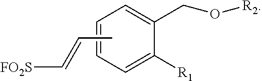

In some embodiments, formula (Ia) is

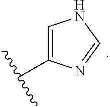 [SMILES: *c1c[nH]cn1]

In some embodiments, the nucleophilic functional group of M is selected from a group consisting of—SH, —NH₂, —SeH, —OH, and

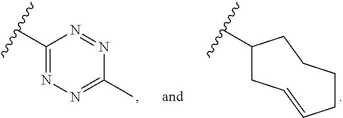

In some embodiments, M is a protein. In some embodiments, M is an antibody.

In some embodiments, the functional molecule in R₂ is a chemical reactive group.

In some embodiments, the functional molecule in R₂ is selected from azido, alkynyl, cyclohydrocarbyl, alkyne, —NH₂, carboxyl, and tetrazine.

In some embodiments, the functional molecule in R₂ is selected from—COOH, —NH₂, —N₃, ethynyl,

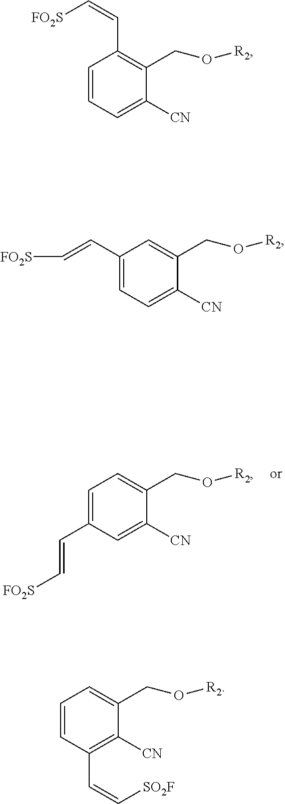

In some embodiments, the method comprises contacting a functional molecule with the conjugate, wherein the functional molecule is selected from a toxin, a fluorescent dye, or a biotin. In some embodiments, the functional molecule is capable of inhibiting the growth of a tumor cell. In some embodiments, R₂ comprise a functional molecule selected from the group consisting of a DNA topoisomerase I inhibitor, a V-ATPase inhibitor, a pro-apoptotic agent, a Bc12 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a nucleoside analog, a HD AC inhibitor; an anthracycline; a NAMPT inhibitor; a hydrophilic prodrug; a SN-38 glucuronide, a etoposide phosphate; a nitrogen mustard, a proteosome inhibitor, a cytokine, a Toll-like receptor agonist and a Sting agonist. In some embodiments, the functional group comprises Dxd.

In some embodiments, the functional molecule is linked with the conjugate via a linker, wherein the linker is selected from the group consisting of a cleavable linker, a non-cleavable linker, a hydrophilic linker, a hydrophobic linker, a procharged linker, an uncharged linker and a dicarboxylic acid-based linker. In some embodiments, the linker is selected from the group consisting of VC-PAB, N-succinimidyl-3-(2-pyridyldithio) propionate (SPDP), N-succinimidyl 4-(2-pyridyldithio) pentanoate (SPP), N-succinimidyl 4-(2-pyridyldithio) butanoate (SPDB), N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl)cyclohexanecarboxylate (SMCC), N-sulfosuccinimidyl 4-(maleimidomethyl)cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

In some embodiments, the method is conducted at a temperature in a range of about 16° C. to about 37° C. For example, the method is conducted at a temperature of about 16° C. or about 37° C.

In some embodiments, the method is conducted at a pH in a range of about 7.4 to about 8.

In another aspect, the present application provides a pharmaceutical composition comprising the conjugate of the present application and optionally a pharmaceutically acceptable carrier.

In another aspect, the present application provides a method for preventing and/or treating tumor in a subject in need of, comprising administering to said subject the conjugate of the present application, or the pharmaceutical composition of the present application.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENC

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates a reaction scheme for synthesizing the arylethenesulfonyl fluorides, and the chemical structures and fields thereof.

FIG. 2 illustrates the LC-MS results of the products from the reaction of SSF derivatives and GFP.

FIG. 3 illustrates a reaction scheme for synthesizing 4-9 (SSF(CN)—O—N₃).

FIG. 4 illustrates a reaction scheme for synthesizing 4-20 (SSF(CN)—O-PEG4-N3-DBCO-PEG4-Dxd).

FIG. 5 illustrates a reaction scheme for synthesizing 4-18 (SSF(CN)—N₃).

FIG. 6 illustrates a reaction scheme for synthesizing 4-21 (SSF(CN)-PEG4-N3-DBCO-PEG4-Dxd).

FIG. 7 illustrates the LC-MS results of the products from the reaction of SSF derivatives and GFP.

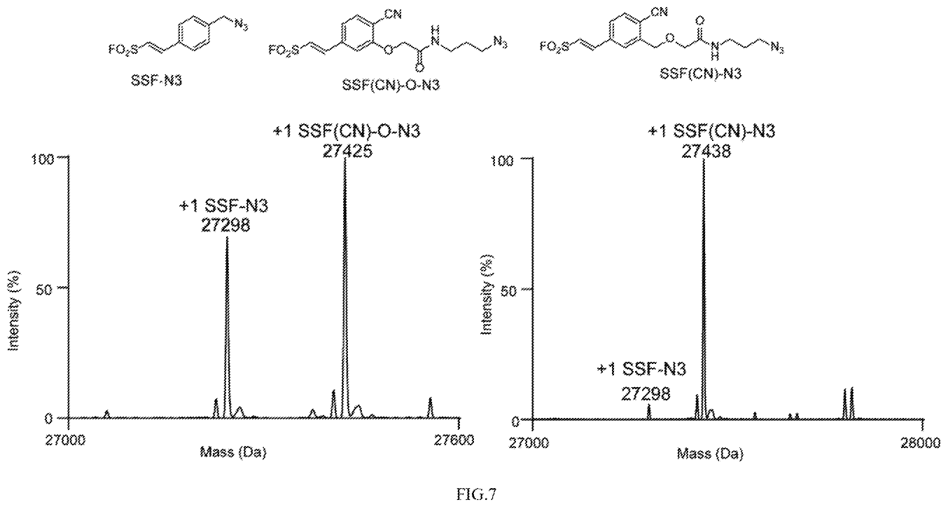

FIG. 8 illustrates a) a reaction scheme for synthesizing the conjugate of the present application, and b) the DAR value of the product from the reaction of SSF derivatives and GFP.

FIG. 9 illustrates a reaction scheme for synthesizing 4-25 (SSF(CN)-PEG4-Dxd).

FIG. 10 illustrates a reaction scheme for synthesizing 4-27 (SSF(CN)-PEG8-Dxd).

FIG. 11 illustrates a reaction scheme for synthesizing 4-29 (SSF(CN)-PEG8-Val-Clt-PAB-MMAE).

FIG. 12 illustrates a reaction scheme for synthesizing 4-31 (SSF(CN)-PEG4-GGFG-Eribuin).

FIG. 13 illustrates a reaction scheme for synthesizing 4-35 (SSF(CN)-PEG8-VC-PAB-Doxorubicin).

FIG. 14 illustrates a) a reaction scheme for synthesizing the conjugate of the present application, and b) the DAR value of the product from the reaction of SSF derivatives and trastuzumab.

FIG. 15 illustrates in vitro activity of conjugate of the present application.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Term

Where substituent groups are specified by their conventional chemical formulae, written from left to right, they equally encompass the chemically identical substituents that would result from writing the structure from right to left, e.g., —CH₂O— is equivalent to —OCH₂—.

The term “conjugate” as used herein generally refers to a any substance formed from the joining together of separate parts. In the conjugate, the separate parts may be joined at one or more active site with each other. Moreover, the separate parts may be covalently or non-covalently associated with, or linked to, each other and exhibit various stoichiometric molar ratios. The conjugate may comprise peptides, polypeptides, proteins, prodrugs which are metabolized to an active agent in vivo, polymers, nucleic acid molecules, small molecules, binding agents, mimetic agents, synthetic drugs, inorganic molecules, organic molecules and radioisotopes. For example, the conjugate may comprise a drug and an antigen binding protein, and may be an antibody drug conjugate, ADC.

The term “antibody” as used herein generally refers to a polypeptide or a protein complex that specifically binds an epitope of an antigen or mimotope thereof. An antibody includes an intact antibody, or a binding fragment thereof that competes with the intact antibody for specific binding and includes chimeric, humanized, fully human, and bispecific antibodies. Binding fragments include, but are not limited to, Fab, Fab′, F(ab′)2, Fv, and single-chain antibodies. In some embodiments, an antibody is referred to as an immunoglobulin and include the various classes and isotypes, such as IgA (IgAQ1 and IgA2), IgD, IgE, IgM, and IgG (IgG1, IgG3 and IgG4) etc. in some embodiments the term “antibody” as used herein refers to polyclonal and monoclonal antibodies and functional fragments thereof. An antibody includes modified or derivatized antibody variants that retain the ability to specifically bind an epitope. Antibodies are capable of selectively binding to a target antigen or epitope. Antibodies may include, but are not limited to polyclonal antibodies, monoclonal antibodies (mAbs), humanized and other chimeric antibodies, single chain antibodies (scFvs), Fab fragments, F(ab′)2 fragments and disulfide-linked Fvs (sdFv) fragments. In some embodiments, the antibody is from any origin, such as mouse or human, including a chimeric antibody thereof. In some embodiments, the antibody is humanized.

The term “derivative” as used herein generally refers to a compound which is expected to exhibit a similar (for example, physical, or/and chemical, or/and biological) activity (ies) as that exhibited by the subject (parent) compound. For example, the derivative may be a precursor, a metabolite, a salt and/or an ester of the subject compound.

The term “alkyl” as used herein generally refers to at least one carbon atoms (For example, 1 to 20 carbon atoms. “1 to 20 carbon atoms” may refer to a straight chain and/or a branched group having an alkyl group of up to 1 carbon atom, 2 carbon atoms, 3 carbon atoms, etc., comprising up to 20 carbon atoms), And saturated aliphatic (i.e., non-aromatic) acyclic hydrocarbons (i.e., groups consisting of carbon atoms and hydrogen atoms) comprising a neighboring carbon-carbon double bond or carbon-carbon triple bond. For example, the alkyl may comprise from 1 to 10 carbon atoms. For example, the alkyl may comprise from 1 to 6 carbon atoms. The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C1-C10 means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “electron withdrawing group” as used herein generally refers to an atom or group that draws electron density from neighboring atoms towards itself, usually by resonance or inductive effects. An electron withdrawing group can be recognized either by the atom adjacent to the π system having several bonds to more electronegative atoms, or, having a formal +ve or δ+ve charge. An electron withdrawing group can remove electron density from the π system making it less nucleophilic. For example, an electron-withdrawing group (EWG) may draw electron away from a reaction center. When this center is an electron rich carbanion or an alkoxide anion, the presence of the electron-withdrawing substituent has a stabilizing effect. Examples of electron withdrawing groups may be halogens (e.g., F, Cl) or —CN.

Each of the above terms (e.g., “alkyl”) includes both substituted and unsubstituted forms of the indicated radical.

In embodiments, each substituted group described in the compounds herein is substituted with at least one substituent group. More specifically, in aspects, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In aspects, at least one or all of these groups are substituted with at least one size-limited substituent group. In aspects, at least one or all of these groups are substituted with at least one lower substituent group.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and(S)—, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “pharmaceutically acceptable carrier” as used herein generally refers to a non-API (API means a pharmaceutically active ingredient) such as disintegrants, binders, fillers, and lubricants used to form a pharmaceutical product. The pharmaceutically acceptable carriers may conform to established government standards, including standards promulgated by the US Food and Drug Administration and the European Food and Drug Administration, and are generally safe for human administration. For example, the pharmaceutically acceptable carrier may comprise a sterile aqueous or non-aqueous solution, a dispersion, a suspension, a emulsion, and/or a sterile injectable solution or dispersion just prior to use.

As used herein, the term “a”, “an”, “the” and similar terms used in the context of the present application (especially in the context of the claims) are to be construed to cover both the singular and plural unless otherwise indicated herein or clearly contradicted by the context.

### Compound

The present application provides a compound of formula (Ib),

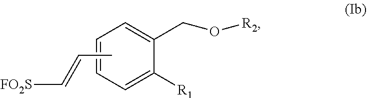

wherein, R₁ is an electron withdrawing group; and R₂ comprises a functional molecule. Compared with the compound of

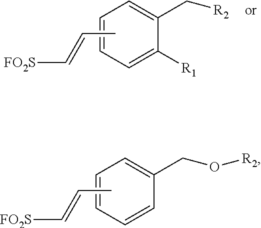

the compound of formula (Ib) has a higher reactivity with a protein.

R₁ may be —CN, —OH, or —CF₃. In a preferable embodiment, the compound is

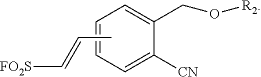

In some embodiments, the functional molecule in R₂ is a chemical reactive group. The functional molecule may be a chemical click group. The functional molecule may be an orthogonal reactive group. The functional molecule may be modified to link a bioactivity molecule, e.g., toxin, or dye.

For example, the functional molecule in R₂ may be selected from azido, alkynyl, cyclohydrocarbyl, alkyne, —NH₂, carboxyl, and tetrazine. For example, the functional molecule in R₂ may be selected from—COOH, —NH₂, —N₃, ethynyl,

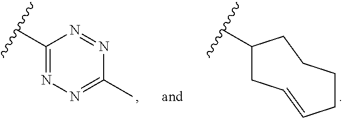

The compound of the present application may be selected from the group consisting of:

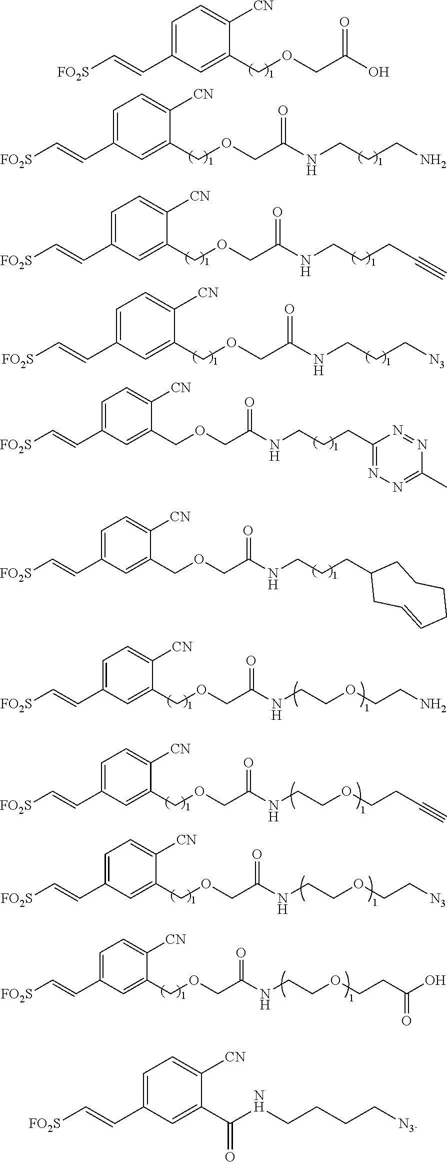

### Conjugate

The compound of the present application has a higher reactivity with antibody to produce an antibody-drug conjugate (ADC). During a same period of time, more amount of the compound of the present application reacts with a protein, e.g., compared with a compound of

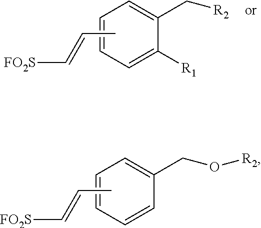

wherein, R₁ is an electron withdrawing group; and R₂ comprises a functional molecule (e.g., a payload).

The present application further provides a conjugate prepared from the compound of the present application. The conjugate of the present application has a formula (I): M-[L]ₐ (I), wherein, M is a biological macromolecule, which is linked to L with a nucleophilic functional group comprised in M; a is an integer of 1 to 8; L is a compound of formula (Ia),

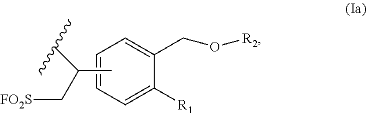

wherein, R₁ is an electron withdrawing group; and R₂ comprises a functional molecule (e.g., a payload).

The conjugate may comprise a functional molecule selected from a toxin, a fluorescent dye, a biotin, or other molecules with interesting activities, after further modification on the chemical reactive group of the compound. The functional molecule may be linked to the antibody via a linker. The linker may be those used in ADC which are known by the skilled person, e.g., a cleavable linker, a non-cleavable linker, a hydrophilic linker, a hydrophobic linker, a procharged linker, an uncharged linker and a dicarboxylic acid-based linker.

For example, the biological macromolecule M may be an antibody. For example, the biological macromolecule M may be an anti-HER2 antibody. For example, the biological macromolecule M may comprise a HCDR1 with the same amino acid sequence as the HCDR1 of Trastuzumab. For example, the biological macromolecule M may comprise a HCDR2 with the same amino acid sequence as the HCDR2 of Trastuzumab. For example, the biological macromolecule M may comprise a HCDR3 with the same amino acid sequence as the HCDR3 of Trastuzumab. For example, the biological macromolecule M may comprise a LCDR1 with the same amino acid sequence as the LCDR1 of Trastuzumab. For example, the biological macromolecule M may comprise a LCDR2 with the same amino acid sequence as the LCDR2 of Trastuzumab. For example, the biological macromolecule M may comprise a LCDR3 with the same amino acid sequence as the LCDR3 of Trastuzumab. For example, the biological macromolecule M may comprise HCDR1, HCDR2 and HCDR3 with the same amino acid sequence as the HCDR1, HCDR2, and HCDR3 of Trastuzumab. For example, the biological macromolecule M may comprise LCDR1, LCDR2 and LCDR3 with the same amino acid sequence as the LCDR1, LCDR2, and LCDR3 of Trastuzumab. For example, the biological macromolecule M may comprise a VH with the same amino acid sequence as the VH of Trastuzumab. For example, the biological macromolecule M may comprise a VL with the same amino acid sequence as the VL of Trastuzumab. For example, the CDR may be defined with Chothia, IMGT or Kabat system.

For example, the biological macromolecule M may be Trastuzumab.

The conjugate of formula M-[L]ₐ may comprise 2, 4, 6, or 8 L, i.e., a is a integer of 2, 4, 6 or 8. The number of the L in the conjugate can be adjusted in different reactive conditions.

The present application further provides a method for preparation the conjugate. The method comprises contacting the compound of formula (Ib) to M, wherein, M is a biological macromolecule, which comprising a nucleophilic functional group; R₁ is an electron withdrawing group; and R₂ comprises a functional molecule. Furthermore, the M (e.g., an antibody) may be conjugated with one or more payloads which is the same with or different from the R₂. For example, the one or more payloads may be conjugated on a glycosylation chain or an amino acid of the antibody.

### Pharmaceutical Composition and Use

In another aspect, the present application provides a pharmaceutical composition comprising the conjugate of the present application and a pharmaceutically acceptable carrier. In present application, the pharmaceutical compositions may comprise the conjugate of the present application presented with a pharmaceutically acceptable carrier. Other pharmacologically active substances may also be present.

In the present application, the conjugate and/or the pharmaceutical composition of the present invention may be administered by any suitable route.

In another aspect, the present application provides a method for adjusting a tumor micro-environment of a subject, comprising administering to the subject the conjugate of the present application, or the pharmaceutical composition of the present application.

In another aspect, the present application provides a method for adjusting the immune reaction of a subject, comprising administering to the subject the conjugate of the present application, or the pharmaceutical composition of the present application.

In another aspect, the present application provides a method for preventing and/or treating disease in a subject in need of, comprising administering to the subject the conjugate of the present application, or the pharmaceutical composition of the present application.

The method may be an in vitro method, an ex vivo method, or an in vivo method. For example, the conjugate of the present application may be administered in vitro to one or more cells. As another example, the conjugate of the present application may be administered to a subject in need thereof.

In another aspect, the present application provides a conjugate of present application, in use of preventing and/or treating disease in a subject in need of.

In another aspect, the present application provides a method of preparing a medicament for treating disease. For example, the disease may be a tumor. For example, the disease may comprise a tumor and/or an autoimmune disease.

For example, the disease may be a HER2-positive tumor. For example, the disease may be a breast cancer.

In another aspect, the present application provides a diagnostic reagent comprising the conjugate of the present application. For example, the label may be selected from the group consisting of a radiolabel, a fluorophore, a chromophore, an imaging agent, and a metal ion.

### Examples

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### LC-MS

LC-MS analysis of protein conjugation and protein-DNA (GFP-20ntssDNA, neo2-20ntssDNA): LC-MS was performed on a Xevo G2-S TOF mass spectrometer coupled to an Acquity high-performance liquid chromatography (UPLC) system using an Acquity UPLC

Protein BEH C4 column (1.7 mm, 2.1×50 mm). Solvents A, water with 0.1% formic acid and B acetonitrile with 0.1% formic acid were used as the mobile phase at a flow rate of 0.5 ml/min. Gradient used: isocratic 95% H₂O for 2 min, then 95% to 10% H₂O in 4 min, then 10% H₂O for 1 min, then 10% to 95% H₂O in 1 min, then 95% H₂O for 2 min. The electrospray source was operated in the positive mode with a capillary with a capillary voltage of 2.0 kV and a cone voltage of 40 V. Nitrogen was used as the desolvation gas at a total flow of 850 L/h. Total mass spectra were reconstructed from the ion series using the MaxEnt algorithm preinstalled on MassLynx software (v. 4.1 from Waters) according to the manufacturer's instructions. To obtain the ion series described, the major peak(s) of the chromatogram were selected for integration and further analysis.

Sequence of GFP:
Engineered GFP is composed of 243 amino
acids and 3 free cysteines
(SEQ ID NO. 1)
MSKGEELFTGVVPILVELDGDVNGHKFSVRGEGEGDATNGKLTLKFICTT
GKLPVPWPTLVTTLTYGVQCFSRYPDHMKRHDFFKSAMPEGYVQERTISF
KDDGTYKTRAEVKFEGDTLVNRICLKGIDFKEDGNILGHKLEYNFNSHNV
YITADKQKNGIKANFKIRHNVEDGSVQLADHYQQNTPIGDGPVLLPDNHY
LSTQSVLSKDPNEKRDHMVLLEFVTAAGITHGGGGLEHHHHHH
Trastuzumab light chain:
(SEQ ID NO. 2)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;
Trastuzumab heavy chain:
(SEQ ID NO. 3)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESLTQPEGKNNTTKPPKV
SNKALPAPIEKTISKSRKQPREPQVYTLPPSREEMTKNQVSLTCLVKSLK
SPK

### Example 1 Synthesis of Arylethenesulfonyl Fluorides

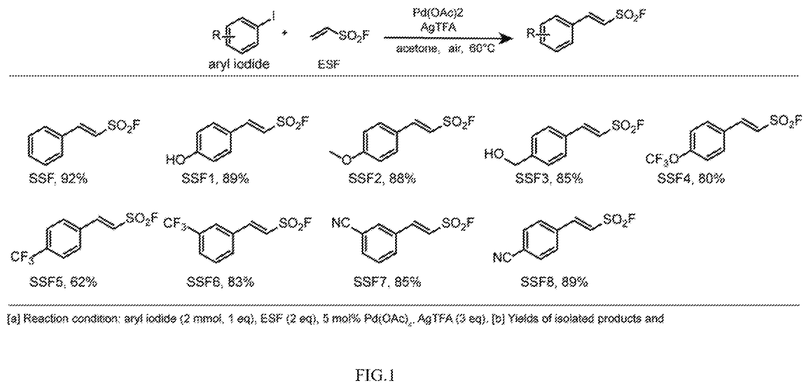

Arylethenesulfonyl fluorides were synthesized based on previously published procedures (Angew. Chem. Int.Ed. 2017, 56, 4849-4852). The reaction condition: aryl iodide (2 mmol, 1 eq), ESF (2 eq), 5 mol % Pd(OAc)₂, and AgTFA (3 eq). Eight arylethenesulfonyl fluorides (named as SSF, SSF1 to SSF8, respectively) were synthesized. The reaction process is shown in FIG. 1.

### Example 2 Competitive Experiment

SSF (1 μL, 5 mM in DMSO), SSF derivative (e.g., SSF1, SSF2, SSF3, SSF4, SSF5, SSF6, SSF7 or SSF8) (1 μL, 5 mM in DMSO), and PBS (14 μL, pH-7.4, 50 mM) were added to a solution of GFP (4 μL, 250 μM in HEPES buffer). After incubation at 37° C. for 1 h, the solution was desalted to give the product before LC-MS analysis.

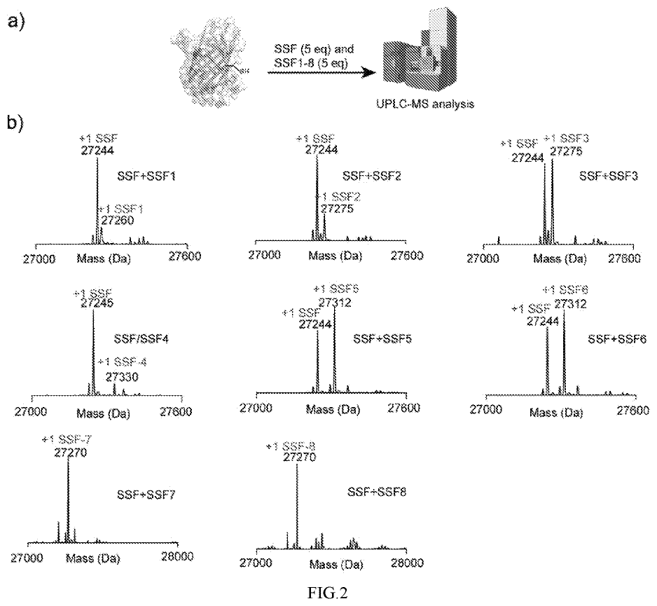

The results were shown in FIG. 2. The reactivity between GFP and the SSF derivatives with hydroxymethyl (SSF3), trifluoromethyl (SSF5, SSF6) and nitrile (SSF7, SSF8), were higher than that between GFP and SSF. The reactivity between GFP and the SSF derivatives with hydroxyl (SSF1), methoxy (SSF2) and trifluoromethoxy (SSF4) were lower than SSF. The hydroxymethyl, trifluoromethyl and nitrile can improve the reactivity of SSF with GFP.

### Example 3 Synthesis of SSF(CF)—O—CF₃ (4-9)

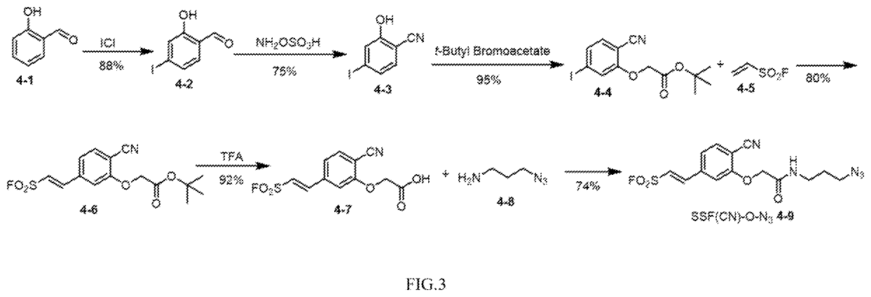

The SSF(CF)—O—CF₃ (4-9) was synthesized from 4-1 according the scheme of FIG. 3.

### Synthesis of 4-2:

An oven-dried reaction tube (250 mL) charged with 4-1 (41 mmol, 5 g), CH₂Cl₂ (20 mL) was added under inert atmosphere. The mixture was cooled to 0° C., and ICI (2 mmol, 1.1 eq.) was dropwisely added. The resulting mixture was incubated at 40° C. for 24 h. The crude material was purified by column chromatography on silica gel to give 4-2. ¹H NMR (400 MHZ, CDCl₃) 7.90 (1H, d, J=7.6 Hz), 7.69-7.65 (1H, m), 7.54-7.48 (2H, m), 5.31 (2H, s); ¹³C NMR (135 MHz, CDCl₃): δ171.1, 146.6, 134.1, 129.0, 125.7, 122.1, 69.7.

### Synthesis of 4-3:

An oven-dried reaction tube (250 mL) charged with 4-2 (20 mmol, 5 g), NH₂OSO₃H (1.1 eq) were added under inert atmosphere. The mixture was cooled to 0° C., and acetic acid (1.1 eq.), H₂O (80 mL) were added. The resulting mixture was incubated at 50° C. for 8 h. The crude material was purified by column chromatography on silica gel to give 4-3. ¹H NMR (400 MHz, DMSO-d): δ 11.36 (1H, s), 7.90 (1H, d, J=1.8 Hz), 7.74 (1H, dd, J=8.8, 1.8 Hz), 6.84 (1H, d, J=8.8 Hz); ¹³C NMR (135 MHz, DMSO-d): δ 159.98, 142.95, 140.50, 118.55, 115.46, 101.47, 80.22.

### Synthesis of 4-4:

An oven-dried reaction tube (20 mL) charged with 4-3 (5 mmol, 1.22 g), K₂CO₃ (1.5 eq) and DMF (80 mL) were added. The mixture was incubated at rt for 30 min, and t-Butyl Bromoacetae (1.2 eq.), was added. The resulting mixture was incubated at 80° C. for 12 h. The crude material was purified by column chromatography on silica gel to give 4-4. ¹H NMR (400 MHz, DMSO-d): δ 7.76 (1H, d, J=2.2 Hz), 7.70 (1H, dd, J=8.9, 2.2 Hz), 6.55 (1H, d, J=8.9 Hz), 4.57 (2H, s), 1.40 (9H, s); ¹³C NMR (135 MHz, DMSO-d): δ166.39, 159.94, 142.78, 141.72, 114.50, 114.45, 104.73, 83.25, 82.36, 66.05, 28.01.

### Synthesis of 4-6:

An oven-dried reaction tube (20 mL) charged with 4-4 (5 mmol, 1.22 g), AgTFA (2.4 mmol, 1.2 eq), Pd(OAc)₂ (22.5 mg, 5 mol %), 4-5 (4 mmol, 2 eq) and acetone (5 mL) were added. The mixture was incubated at 60° C. for 12 h. The crude material was purified by column chromatography on silica gel to give 4-6.

### Synthesis of 4-7:

An oven-dried reaction tube (20 mL) charged with 4-6 (171 mg, 0.5 mmol), TFA (2 mL), and CH₂Cl₂ (2 mL) were added. The resulting mixture was incubated at rt for 4 h. The crude material was purified by column chromatography on silica gel to give 4-7. ¹H NMR (400 MHz, DMSO-d): δ 8.35 (1H, d, J=2.0 Hz), 8.14 (1H, dd, J=9.0, 2.0 Hz), 8.00 (1H, d, J=15.4 Hz), 7.86 (1H, dd, J=15.4, 2.0 Hz), 7.34 (1H, d, J=9.0 Hz), 5.03 (2H, s); 13C NMR (135 MHz, DMSO-d): δ162.48, 147.52, 136.70, 135.90, 125.28, 118.61, 118.35, 115.92, 114.41, 102.05, 65.83; ¹⁹F (376 MHz, DMSO-d): δ+63.18.

### Synthesis of 4-9:

An oven-dried reaction tube (20 mL) charged with 4-7 (71 mg, 0.25 mmol), 4-8 (1 eq), HOBt (1.5 eq), EDC (1.5 eq) and DIPEA (3 eq), dry DMF (2 mL) were added under inert atmosphere. The resulting mixture was incubated at rt for 12 h. The crude material was purified by column chromatography on silica gel to give 4-9. ¹H NMR (400 MHz, DMSO-d): δ 8.35 (1H, d, J=2.2 Hz), 8.21-8.11 (2H, m), 7.98 (1H, d, J=15.4 Hz), 7.85 (1H, dd, J=15.4, 2.2 Hz), 4.82 (2H, s), 3.36-3.30 (2H, m), 3.21-3.16 (2H, m), 1.71-1.64 (2H, m); ¹³C NMR (135 MHZ, DMSO-d): δ 166.8, 162.7, 147.5, 136.7, 135.8, 125.3, 118.6, 118.3, 115.9, 114.2, 102.4, 68.0, 48.8, 36.28, 28.7; ¹⁹F NMR (376 MHZ, CDCl₃): δ+63.1.

### Example 4 Synthesis of SSF(CN)—O-PEG4-N3-DBCO-PEG4-Dxd (4-20)

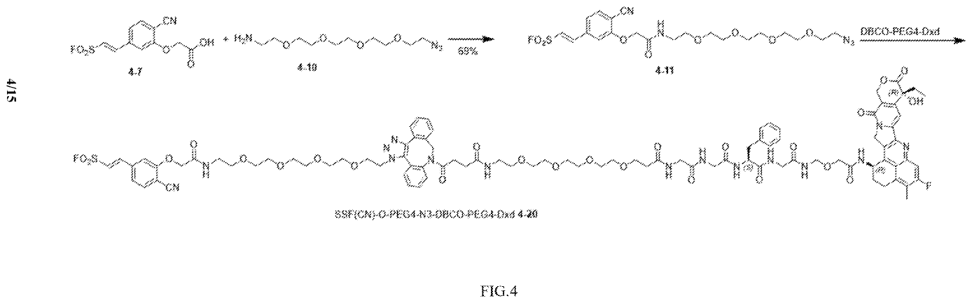

The SSF(CF)—O-PEG4-N3-DBCO-PEG4-Dxd (4-20) was synthesized from 4-7 and 4-10 according the scheme of FIG. 4.

### Synthesis of 4-11:

An oven-dried reaction tube (20 mL) charged with 4-7 (71 mg, 0.25 mmol), 4-10 (1 eq mmol), HOBt (1.5 eq), EDC (1.5 eq) and DIPEA (2 eq), dry DMF (2 mL) were added under inert atmosphere. The resulting mixture was incubated at rt for 12 h. The crude material was purified by column chromatography on silica gel to give 4-11. ¹H NMR (400 MHZ, CDCl₃): δ 7.85-7.84 (1H, m), 7.77 (1H, dd, J=8.8, 2.0 Hz), 7.70 (1H, d, J=15.4 Hz), 7.15 (1H, s), 7.07 (1H, d, J=8.8 Hz), 6.89 (1H, dd, J=15.4, 2.0 Hz), 4.67 (2H, s), 3.64-3.52 (18H, m), 3.36 (2H, t, J=5.2 Hz); ¹³C NMR (100 MHz, CDCl₃): 166.0, 161.28, 145.53, 135.26, 134.37, 125.31, 118.99, 118.70, 114.63, 113.53, 103.85, 70.6, 70.56, 70.47, 70.36, 69.94, 69.35, 68.0, 50.65, 39.11. ¹⁹F (376 MHZ, CDCl₃): δ +62.40.

### Synthesis of 4-20:

Tube (1.5 mL) charged with 4-11 (20 μL, 50 mM in DMSO) and DBC-GGFG-Dxd (20 μL, 50 mM in DMSO) were added. The resulting mixture was incubated at 60° C. for 12 h to obtain 4-20.

### Example 5 Synthesis of SSF(CN)—N3 (4-18)

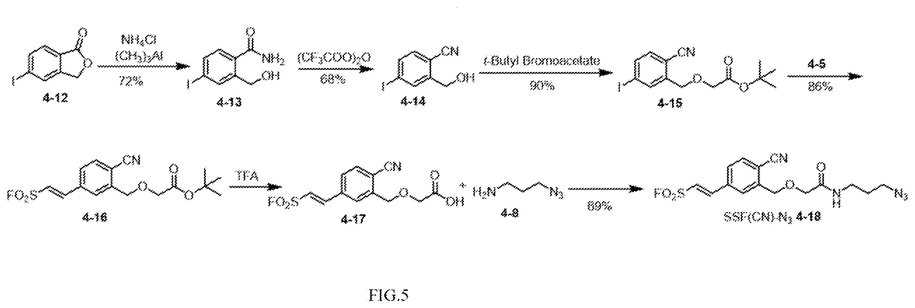

The SSF(CN)—N3 (4-18) was synthesized from 4-12 according the scheme of FIG. 5.

### Synthesis of 4-13:

An oven-dried reaction tube (100 mL) charged with NH₄Cl (15 mmol, 3 eq), THF (10 mL) were added under inert atmosphere. The mixture was cooled to 0° C., and 7.5 mL (15.0 mmol) of (CH₃)₃Al (2.0 M in Toluene) was dropwise added to the tube. The resulting mixture was incubated at rt for 2 h, and 4-12 (1.3 eq) was added. The resulting mixture was incubated at 50° C. for 18 h. The crude material was purified by column chromatography on silica gel to give 4-13. ¹H NMR (400 MHz, d-DMSO): δ 7.91-7.88 (2H, m), 7.68 (1H, dd, J=8.0, 1.82 Hz), 7.46 (1H, s), 7.25 (1H, d, J=8.0 Hz), 5.38-5.36 (1H, t, J=5.6 Hz), 4.60 (2H, d, J=5.6 Hz); 13C NMR (100 MHz, CDCl₃): 170.2, 143.4, 136.2, 135.6, 134.5, 129.8, 97.3, 60.8; HRMS m/z (ESI): calcd for C₈H₈INO₂ [M+H]⁺: 270.9600, found 270.3563.

### Synthesis of 4-14:

An oven-dried reaction tube (100 mL) charged with 4-13 (2.7 g, 10 mol, 1 eq), Py (4 eq) and CH₂Cl₂ (20 mL) were added under inert atmosphere. The mixture was cooled to 0° C., and TFA anhydride (2 eq) was dropwise added to the tube. The resulting mixture was incubated at rt for 12 h. The crude material was purified by column chromatography on silica gel to give 4-14. ¹H NMR (400 MHZ, CDCl₃): δ 8.04 (1H, s), 7.75 (1H, dd, J=8.0, 1.8 Hz), 7.34 (1H, d, J=8.0 Hz), 4.86 (2H, s); 13C NMR (100 MHZ, CDCl₃): 145.7, 137.2, 137.0, 133.5, 116.8, 109.7, 100.9, 62.1. HRMS m/z (ESI): calcd for C₈H₇INO [M+H]⁺: 259.9572, found 259.8564.

### Synthesis of 4-15:

An oven-dried reaction tube (100 mL) charged with 4-14 (5 mol, 1.29 g, 1 eq), t-Butyl Bromoacetae (4 eq), TBAB (0.5 eq), toluene (15 mL) and 5 mol/L (15 mL). The resulting mixture was incubated at rt for 24 h. The crude material was purified by column chromatography on silica gel to give 4-15.

¹H NMR (400 MHZ, CDCl₃): δ 8.06 (1H, s), 7.74 (1H, d, J=7.8 Hz), 7.32 (1H, d, J=8.0 Hz), 4.75 (2H, s), 4.10 (2H, s), 1.49 (9H, s); ¹³C NMR (135 MHZ, CDCl₃): δ168.9,142.8, 137.7, 137.4,133.3, 116.7, 110.3, 100.7, 82.2, 69.9, 68.7, 28.1; HRMS m/z (ESI): calcd for C₁₄H₁₇INO₃ [M+H]⁺: 374.0253, found 374.0346.

### Synthesis of 4-16:

An oven-dried reaction tube (20 mL) charged with 4-15 (5 mmol, 748 mg), AgTFA (2.4 mmol, 1.2 eq), Pd(OAc)₂ (22.5 mg, 5 mol %), 4-5 (4 mmol, 2 eq) and acetone (5 mL) were added. The mixture was incubated at 80° C. for 12 h. The crude material was purified by column chromatography on silica gel to give 4-16. ¹H NMR (400 MHZ, CDCl₃): δ 7.96 (1H, s), 7.82 (1H, d, J=15.4 Hz), 7.74 (1H, d, J=7.8 Hz), 7.56 (1H, d, J=7.4 Hz), 7.04 (1H, dd, J=15.4, 2.2 Hz), 4.86 (2H, s), 4.16 (2H, s), 1.51 (9H, s); 13C NMR (135 MHZ, CDCl₃): δ168.5, 142.6, 138.1, 137.6, 133.0, 124.4, 118.6, 116.9, 110.4, 100.7, 81.8, 70.1, 68.8, 28.3; ¹⁹F (376 MHz, CDCl3): δ +61.98; HRMS m/z (ESI): calcd for C₁₆H₁₉FNO₅S [M+H]⁺: 356.0968, found 356.1034.

### Synthesis of 4-17:

An oven-dried reaction tube (20 mL) charged with 4-16 (356 mg, 1 mmol), TFA (4 mL), and CH₂Cl₂ (4 mL) were added. The resulting mixture was incubated at rt for 4 h. The crude material was purified by column chromatography on silica gel to give 4-17.

### Synthesis of 4-18:

An oven-dried reaction tube (20 mL) charged with 4-17 (44.8 mg, 0.15 mmol), 4-8 (1 eq), HOBt (1.5 eq), EDC (1.5 eq) and DIPEA (3 eq), dry DMF (2 mL) were added under inert atmosphere. The resulting mixture was incubated at rt for 12 h. The crude material was purified by column chromatography on silica gel to give 4-18. ¹H NMR (400 MHZ, CDCl₃): δ 7.93-7.63 (4H, m), 7.07 (1H, d, J=15.4 Hz), 6.88 (1H, s), 4.79 (2H, s), 4.10 (2H, s), 3.41-3.81 (4H, m), 2.98-2.86 (2H, m); ¹³C NMR (135 MHz, CDCl₃): δ 168.7, 145.9, 141.7, 134.1, 129.0, 128.8, 122.4, 122.1, 116.6, 114.8, 70.9, 70.4, 49.4, 36.7, 28.6; ¹⁹F (376 MHz, CDCl₃): δ +61.95; HRMS m/z (ESI): calcd for C₁₅H₁₇FN₅O₄S [M+H]⁺: 382.0985, found 382.0178.

### Example 6 Synthesis of SSF(CN)-PEG4-N3-DBCO-PEG4-Dxd (4-21)

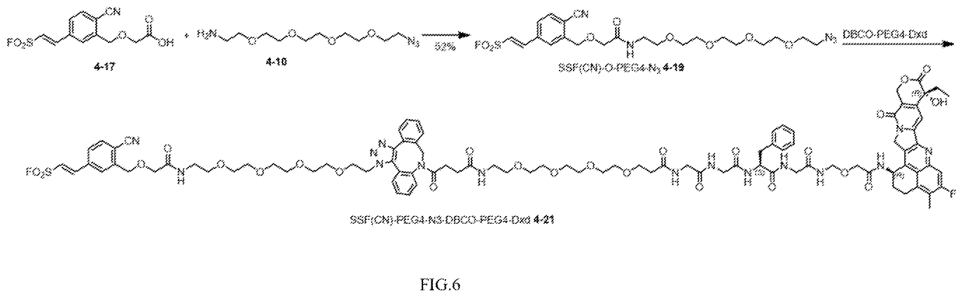

The SSF(CN)-PEG4-N3-DBCO-PEG4-Dxd (4-21) was synthesized from 4-17 and 4-10 according the scheme of FIG. 6.

### Synthesis of 4-19:

An oven-dried reaction tube (20 mL) charged with 4-17 (74.7 mg, 0.25 mmol), 4-10 (1.1 eq mmol), HOBt (1.5 eq), EDC (1.5 eq) and DIPEA (2 eq), dry DMF (2 mL) were added under inert atmosphere. The resulting mixture was incubated at rt for 12 h. The crude material was purified by column chromatography on silica gel to give 4-19. ¹H NMR (400 MHZ, CDCl₃): δ 7.84-7.76 (4H, m), 7.61 (1H, dd, J=8.0 1.6 Hz), 7.13 (1H, dd, J=15.8 2.4 Hz), 7.02-7.00 (1H, m), 4.79 (2H, s), 4.10 (2H, s), 3.67-3.57 (16H, m), 3.52-3.48 (2H, m), 3.36-3.33 (2H, m); ¹³C NMR (135 MHz, CDCl₃): δ 168.5, 145.9, 142.0, 135.4, 133.8, 128.7, 122.5, 122.2, 116.2, 114.6, 70.6, 70.5, 70.4, 70.1, 70.0, 69.5, 50.6, 38.7; ¹⁹F (376 MHZ, CDCl₃): δ+61.90; HRMS m/z (ESI): calcd for C₂₂H₃₁FN₅O₈S [M+H]⁺: 357.2502, found 357.2563.

### Synthesis of 4-21:

Tube (1.5 mL) charged with 4-19 (20 μL, 50 mM in DMSO) and DBC-GGFG-Dxd (20 μL, 50 mM in DMSO) were added. The resulting mixture was incubated at 60° C. for 12 h to obtain 4-21.

### Example 7 Competitive Experiment

SSF—N3 (1 μL, 5 mM in DMSO), SSF(CN)—O—N₃ or SSF(CN)—N₃ (1 μL, 5 mM in DMSO), and PBS (14 μL, pH=7.4, 50 mM) were added to a solution of GFP (4 μL, 250 μM in HEPES buffer). After incubation at 37° C. for 1 h, the solution was desalted to give the product before LC-MS analysis.

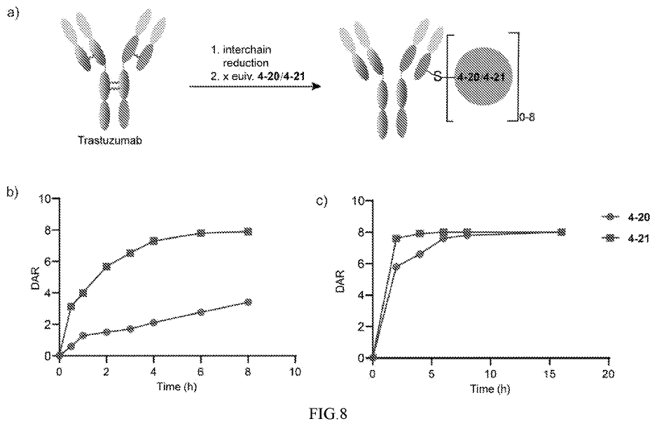

The results were shown in FIG. 8. The reactivity of SSF(CN)—O—N₃ and SSF(CN)—N₃ with GFP are higher than the reactivity of SSF—CN₃ with GFP. And SSF(CN)—N₃ has the highest reactivity with GFP.

### Example 8 Kinetic Experiment

4-20 or 4-21 (1 μL, 10.4 mM in DMSO) and PBS (75 μL, pH=7.4, 50 mM) was added to a solution of trastuzumab (4 μL, 130 μM, HEPES buffer). After incubation at 16/37° C. for various times, the solution was desalted to give the product before LC-MS analysis.

The reaction kinetics of Trastuzumab with variation equivalent 4-20/4-21 (a) and the DAR value of the products incubated at 16° C. (b) and 37° C. (c) shown in FIG. 8. The results show that, after reaction of 20 eq of the compound 4-21 and the antibody at 16° C. for 8 hours, antibody-drug conjugate with a DAR value of 8 is produced, while after reaction of 20 eq of the compound of 4-20 and the antibody at 16° C. for 8 hours, the DAR value of the ADC product is 3.6. And at 37° C., the reactivity of 4-21 with the antibody is higher than that of 4-20 with the antibody.

### Example 9 Synthesis of SSF(CN)-PEG4-Dxd (4-25)

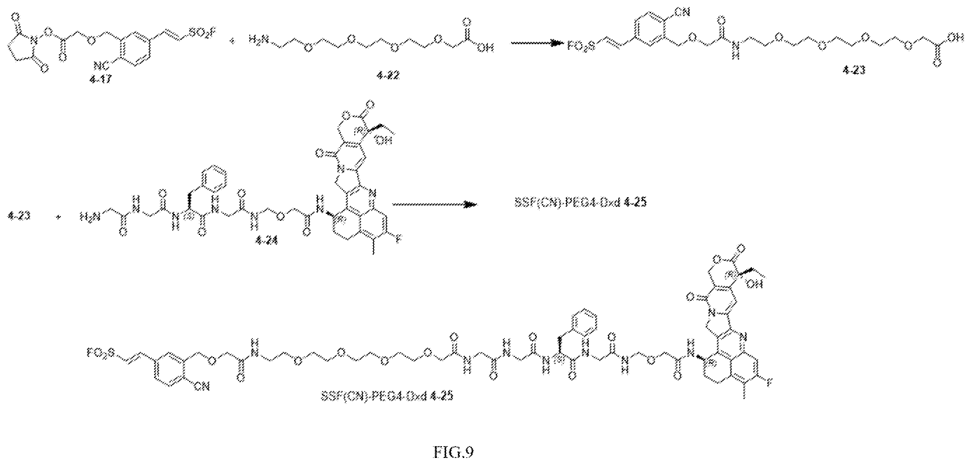

The SSF(CN)-PEG4-Dxd (4-25) was synthesized from 4-17 and 4-22 according the scheme of FIG. 9.

### Synthesis of 4-23:

To a mixture of 4-17 (3 mg, 1.0 eq) and 4-22 (1.9 mg, 1.0 eq) in CH₂Cl₂ (1.5 mL), were added DIPEA (1.5 eq). The solution was stirred at rt for 3 h until the reaction was finished as indicated by TLC and MS. The solution was diluted with CH₂Cl₂, and the organic layer was washed with brine (3 times), dried over MgSO₄, concentrated and purified with CombiFlash to give 4-23 (3.8 mg, 95%). ¹H NMR (400 MHZ, CDCl₃): δ 8.05 (1H, s), 7.85 (3H, m), 7.62 (1H, dd, J=8.2, 2.0 Hz), 7.20 (1H, m), 4.83 (2H, s), 4.15 (2H, s), 4.11 (2H, s), 3.71 (2H, m), 3.67-3.59 (12H, m), 3.53 (2H, d, J=15.7 Hz); ¹³C NMR (135 MHz, CDCl₃): δ 171.1, 168.8 (CO), 145.9, 141.9, 133.6, 130.1, 128.7, 125.9, 122.7, 122.3, 116.2, 70.6, 70.5, 70.4, 70.3, 70.3, 70.1, 70.0, 69.5, 69.0, 53.4, 38.7; ¹⁹F (376 MHz, CDCl₃): δ+62.40. HRMS m/z (ESI): Calcd for C₂₂H₃₀FN₂O₁₀S [M+H]+: 532.1527, found 532.5353.

### Synthesis of 4-25:

To a mixture of 4-23 (3 mg, 1.0 eq) and 4-24 (4.7 mg, 1.0 eq) in DMF (2 mL), were added HATu (1.5 eq). HOBt (1.5 eq) and DIPEA (3 eq). The solution was stirred at rt for 4 h until the reaction was finished as indicated by TLC and MS. The solution was diluted with CH₂Cl₂/H₂O (10 mL/2 mL), and the organic layer was washed with brine (3 times), dried over MgSO₄, concentrated and purified with CombiFlash to give 4-25 (6 mg, 80%). HRMS m/z (ESI): Calcd for C₆₄H₇₃F₂N₁₀O₁₉S [M+H]⁺: 1355.4742, found 1355.4532; Calcd for C₆₄H₇₂F₂N₁₀NaO₁₉S [M+Na]⁺: 1377.4562, found 1377.4495.

### Example 10 Synthesis of SSF(CN)-PEG8-Dxd (4-27)

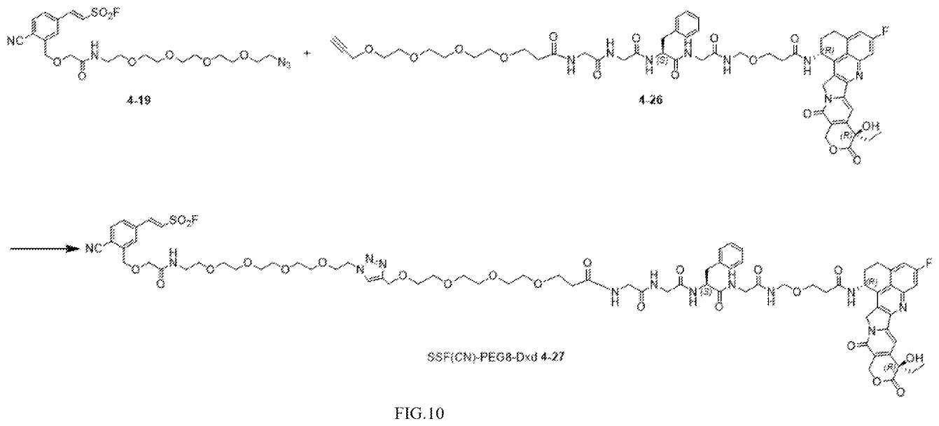

The SSF(CN)-PEG8-Dxd (4-27) was synthesized from 4-19 and 4-26 according the scheme of FIG. 10.

### Synthesis of 4-27:

To a mixture of 4-19 (3 mg, 1.0 eq) and 4-26 (6.0 mg, 1.0 eq) in CH₂Cl₂/H₂O (2 mL/0.5 mL), were added CuSO₄5H₂O (0.5 eq) and Na ascorbate (1.0 eq). The solution was stirred at rt for 12 h until the reaction was finished as indicated by TLC and MS. The solution was diluted with CH₂Cl₂, and the organic layer was washed with brine (3 times), dried over MgSO₄, concentrated and purified with CombiFlash to give 4-27 (6.1 mg, 70%). HRMS m/z (ESI): Calcd for C₇₆H₉₄F₂N₁₃O₂₃S [M+H]⁺: 1626.6274, found 1626.6115; Calcd for C₇₆H₉₃F₂N₁₃NaO₂₃S [M+Na]⁺: 1648.6094, found 1648.5905.

### Example 11 Synthesis of SSF(CN)-PEG8-Val-Cit-PAB-MMAE (4-29)

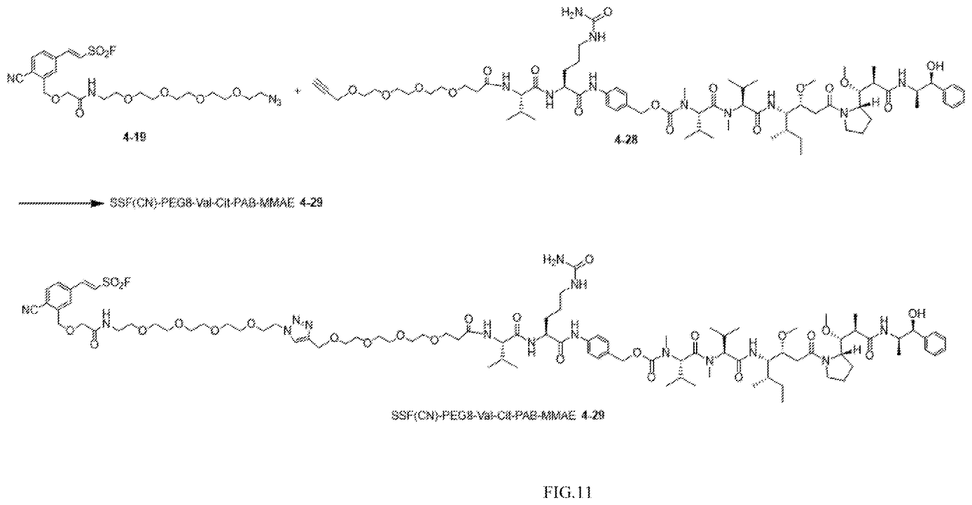

The SSF(CN)-PEG8-Val-Cit-PAB-MMAE (4-29) was synthesized from 4-19 and 4-28 according the scheme of FIG. 11.

### Synthesis of 4-29:

To a mixture of 4-19 (2 mg, 1.0 eq) and 4-28 (6.0 mg, 1.0 eq) in CH₂Cl₂/H₂O (2 mL/0.5 mL), were added CuSO₄5H₂O (0.5 eq) and Na ascorbate (1.0 eq). The solution was stirred at rt for 12 h until the reaction was finished as indicated by TLC and MS. The solution was diluted with CH₂Cl₂, and the organic layer was washed with brine (3 times), dried over MgSO₄, concentrated and purified with CombiFlash to give 4-29 (5.3 mg, 76%). HRMS m/z (ESI): calcd for C₉₂H₁₄₃FN₁₅O₂₅S [M+H]⁺: 1909.0084, found 1909.0062; Calcd for C₉₂H₁₄₂FN₁₅NaO₂₅S [M+Na]⁺: 1930.9904, found 1930.9756.

### Example 12 Synthesis of SSF(CN)-PEG4-GGFG-Eribulin (4-31)

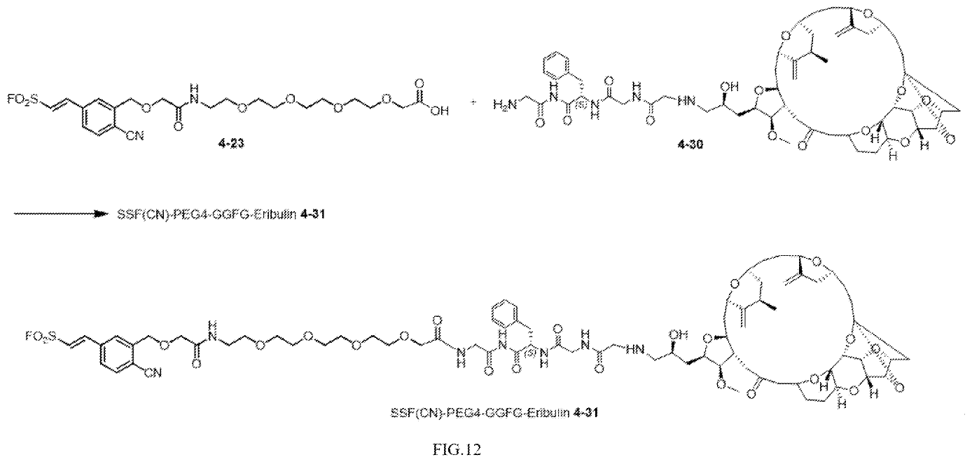

The SSF(CN)-PEG8-Dxd (4-27) was synthesized from 4-23 and 4-30 according the scheme of FIG. 12.

### Synthesis of 4-31:

To a mixture of 4-23 (1.5 mg, 1.0 eq) and 4-30 (3 mg, 1.0 eq) in DMF (2 mL), were added HATu (1.5 eq). HOBt (1.5 eq) and DIPEA (3 eq). The solution was stirred at rt for 4 h until the reaction was finished as indicated by TLC and MS. The solution was diluted with CH₂Cl₂/H₂O (10 mL/2 mL), and the organic layer was washed with brine (3 times), dried over MgSO₄, concentrated and purified with CombiFlash to give 4-31 (3.3 mg, 75%). HRMS m/z (ESI): Calcd for C₇₇H₁₀₅FN₇O₂₄S [M+H]⁺: 1562.6916, found 1562.6921; Calcd for C₇₇H₁₀₄FN₇NaO₂₄S [M+Na]⁺: 1584.6735, found 1584.6599.

### Example 13 Synthesis of SSF(CN)-PEG8-VC-PAB-Doxorubicin (4-35)

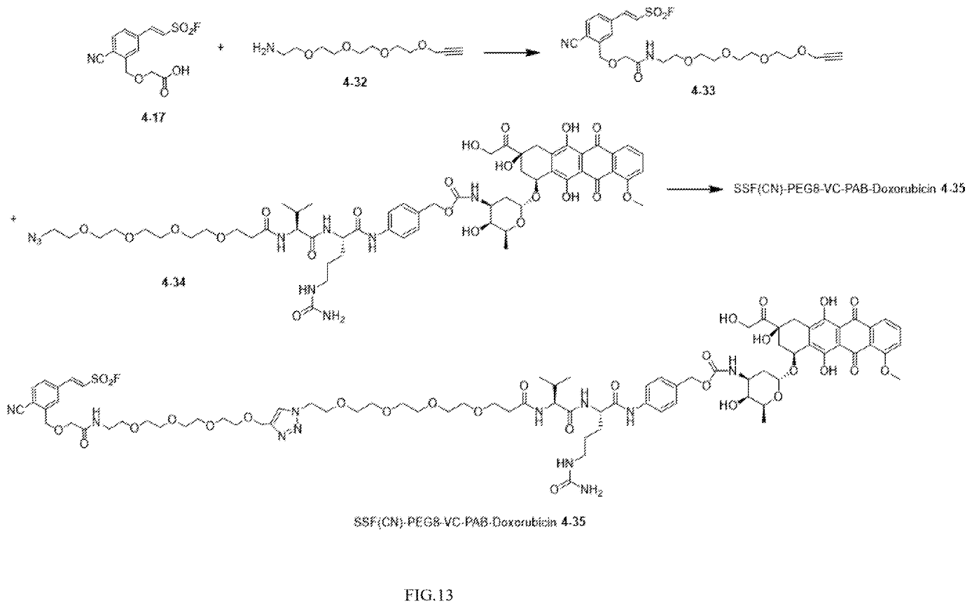

The SSF(CN)-PEG8-VC-PAB-Doxorubicin (4-35) was synthesized from 4-17 and 4-32 according the scheme of FIG. 13.

### Synthesis of 4-33:

To a mixture of 4-17 (3 mg, 1.0 eq) and 4-32 (2.4 mg, 1.0 eq) in DMF (2 mL), were added HATu (1.5 eq). HOBt (1.5 eq) and DIPEA (3 eq). The solution was stirred at rt for 4 h until the reaction was finished as indicated by TLC and MS. The solution was diluted with CH₂Cl₂/H₂O (10 mL/2 mL), and the organic layer was washed with brine (3 times), dried over MgSO₄, concentrated and purified with CombiFlash to give 4-33 (4.6 mg, 85%). ¹H NMR (400 MHz, CDCl₃): δ 8.10 (1H, s), 7.89 (1H, s), 7.81 (3H, m), 7.67 (1H, dd, J=15.8, 2.0 Hz), 7.14 (1H, m), 4.82 (2H, s), 4.13 (2H, s), 4.10 (2H, s), 3.66 (2H, m), 3.65-3.58 (12H, m), 3.53 (2H, d, J=15.4 Hz); ¹⁹F (376 MHz, CDCl₃): δ +62.40. ¹³C NMR (135 MHz, CDCl₃): δ 169.4 (CO), 146.2, 142.1, 135.5, 133.8, 128.7, 125.9, 122.5, 122.2, 116.2, 77.5, 76.8, 71.1, 70.6, 70.5, 70.4, 70.3, 70.3, 70.2, 70.2, 69.8, 69.0, 38.8; HRMS m/z (ESI): Calcd for C₂₂H₃₀FN₂O₁₀S [M+H]⁺: 513.1707, found 513.1912; Calcd for C₂₂H₂₉FN₂NaO₁₀S [M+Na]⁺: 535.1526, found 535.1205.

### Synthesis of 4-35:

To a mixture of 4-19 (3 mg, 1.0 eq) and 4-28 (7.1 mg, 1.0 eq) in CH₂Cl₂/H₂O (2 mL/0.5 mL), were added CuSO₄5H₂O (0.5 eq) and Na ascorbate (1.0 eq). The solution was stirred at rt for 12 h until the reaction was finished as indicated by TLC and MS. The solution was diluted with CH₂Cl₂, and the organic layer was washed with brine (3 times), dried over MgSO₄, concentrated and purified with CombiFlash to give 4-31 (5.3 mg, 53%). HRMS m/z (ESI): calcd for C₉₂H₁₄₃FN₁₅O₂₅S [M+H]⁺: 1909.0084, found 1910.0677; Calcd for C₉₂H₁₄₂FN₁₅NaO₂₅S [M+Na]⁺: 1930.9904, found 1931.2200. HRMS m/z (ESI): Calcd for C₈₀H₁₀₅FN₁₁O₂₉S [M+H]⁺: 1734.6784, found 1734.6554; Calcd for C₈₀H₁₀₄FN₁₁NaO₂₉S [M+Na]⁺: 1756.6604, found 1756.6295.

### Example 14 Conjuagtes Experiment

4-20, 4-21, 4-25, 4-27, 4-29, 4-35 or 4-35 (1 μL, 10.4 mM in DMSO) and PBS (75 μL, pH=7.4, 50 mM) was added to a solution of trastuzumab (4 μL, 130 μM, HEPES buffer). After incubation at 37° C. for 2.5 h, the solution was desalted to give the product before LC-MS analysis.

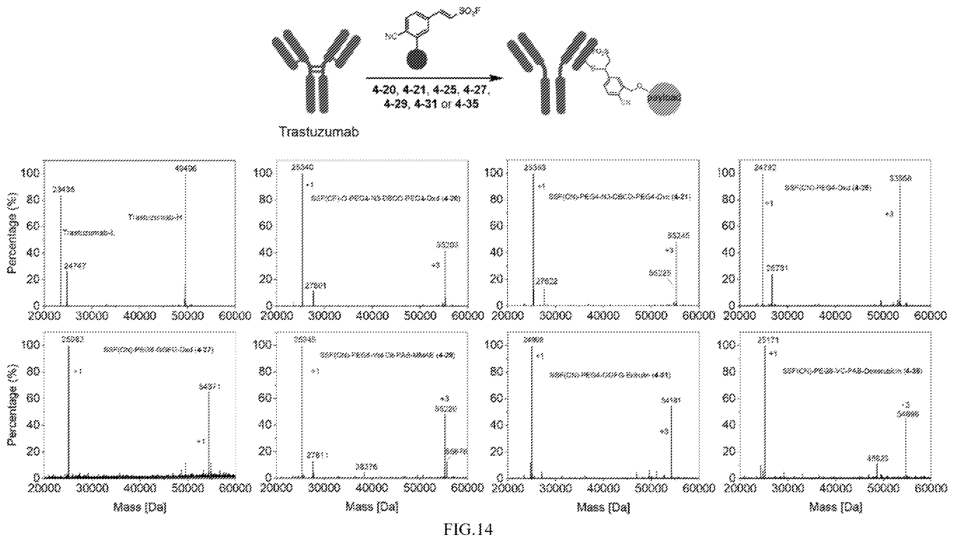

The results were shown in FIG. 14.

### Example 15 In Vitro and In Vivo Activity of Conjugate

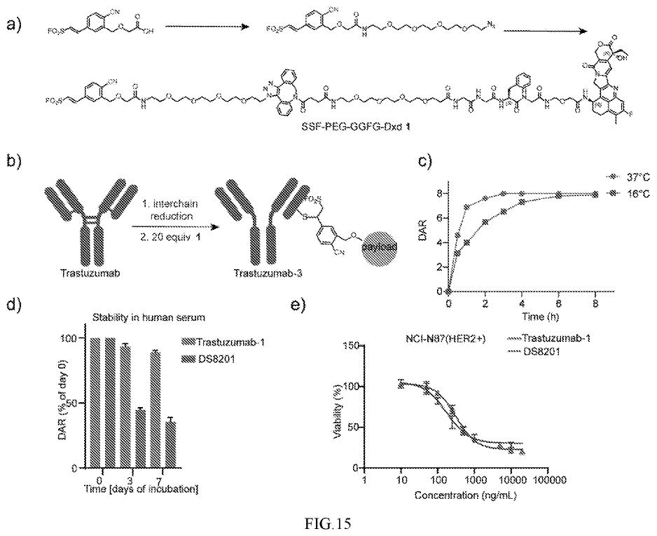

Conjugates obtained from example 14 were incubated with breast cancer cell line, e.g., NCI-N87. Trastuzumab-3 and DS8201 (an anti-HER-2 ADC) were used for FIG. 15 d) serum stability and FIG. 15 e) cell killing experiments. FIG. 15 d) DS8201 and trastuzumab-3 were incubated in human serum for 3 days and 7 days at 37° C., respectively. And then DS8201 and trastuzumab-3 were purified by Protein A, and DAR of ADCs were detected by mass spectrometry; FIG. 15 e) To the 96-well plates were inoculated with NCI-N87 at 37° C. and 5% carbon dioxide for 24 hours, the medium was removed. To each well was added 100 μL of trastuzumab-3 and DS8201 medium containing different dilutions, and the PBS group was used as the control well. And 96-well plates was cultured at 37° C. and 5% carbon dioxide for 72 h. Finally, cell viability was measured. The results of in-vitro experiment shows that the conjugates are able to inhibit cell growth. The results in FIG. 15 shows that the conjugate from 4-21 is more stable than DS8201 (an anti-HER-2 ADC) in human serum and have a comparable ability to kill HER-2 positive cell line NCI-N87.

Conjugate obtained from example 14 were administrated to mice model with breast cancer. The results of in-vivo experiment shows that the conjugates are able to inhibit tumor growth of mice model.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A compound of formula (Ib), 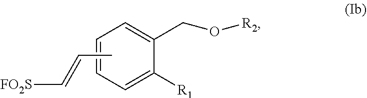
wherein,
R₁ is an electron withdrawing group selected from —CN, —NO₂, —CHO, fluoro, trifluoro methyl, acetoxy, sulfonic acid, —COR₁₁, and —COOR₁₂, wherein, each of R₁₁ and R₁₂ is independently selected from C₁-C₆ alkyl; and
R₂ comprises a functional molecule selected from azido, alkynyl, cyclohydrocarbyl, alkyne, —NH₂, carboxyl, and tetrazine.

2. (canceled)

3. The compound of claim 1, wherein R₁ is —CN.

4. The compound of claim 1, which is

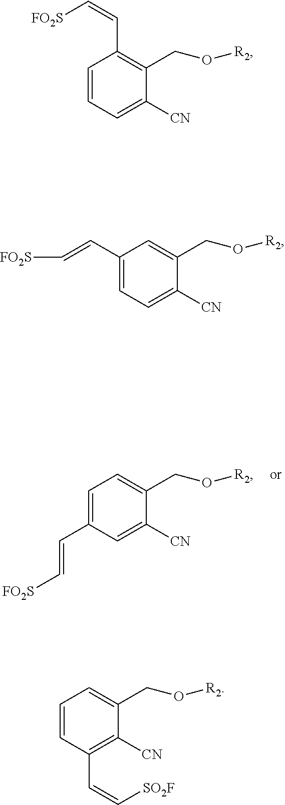

5. (canceled)

6. (canceled)

7. The compound of claim 1, wherein the functional molecule in R₂ is selected from —COOH, —NH₂, —N₃, ethynyl,

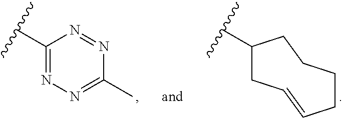

8. The compound of claim 1, which is selected from the group consisting of: 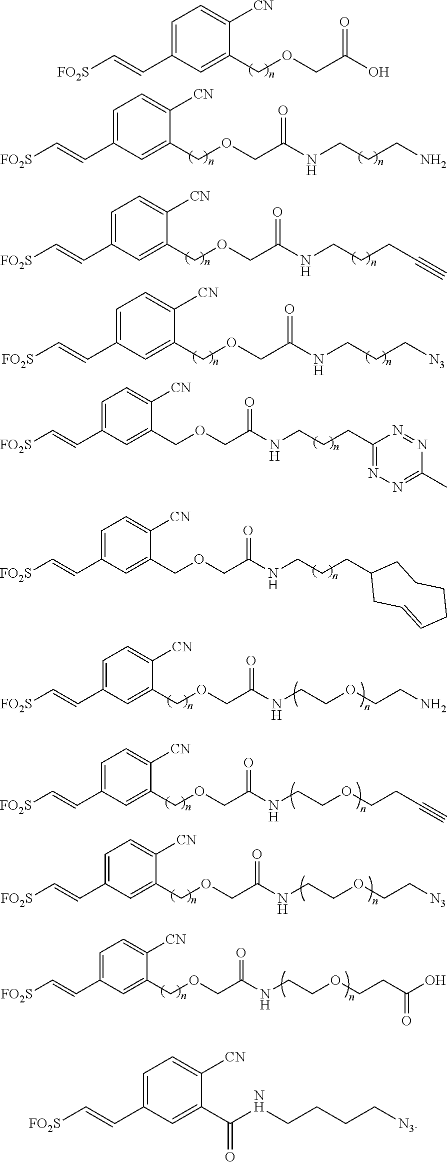
wherein each of n is an integer selected from 1-8.

9. A conjugate of formula (I),
M-[L]ₐ (I), wherein,
M is a protein, which is linked to L with a nucleophilic functional group comprised in M;
a is an integer selected from 1 to 8;
L is a compound of formula (Ia), 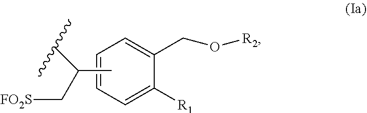
wherein,
R₁ is an electron withdrawing group selected from —CN, —NO₂, —CHO, fluoro, trifluoro methyl, acetoxy, sulfonic acid, —COR₁₁, and —COOR₁₂, wherein, each of R₁₁ and R₁₂ is independently selected from C₁-C₆ alkyl; and
R₂ comprises a functional molecule selected from azido, alkynyl, cyclohydrocarbyl, alkyne, —NH₂, carboxyl, and tetrazine, or
R₂ comprise a functional molecule selected from a toxin, a fluorescent dye, or a biotin.

10. (canceled)

11. The conjugate of claim 9, wherein R₁ is —CN.

12. The conjugate of claim 9, wherein L is

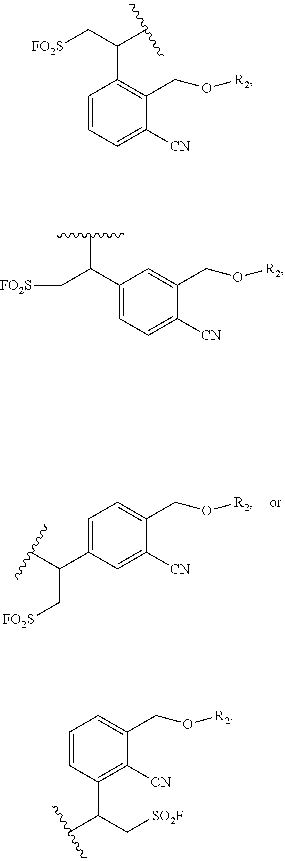

13. The conjugate of claim 9, wherein said nucleophilic functional group of M is selected from a group consisting of —SH, —NH₂, —SeH, —OH, and

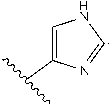 [SMILES: *c1c[nH]cn1]

14. (canceled)

15. (canceled)

16. (canceled)

17. (canceled)

18. The conjugate of claim 9, wherein the functional molecule in R₂ is selected from —COOH, —NH₂, —N₃, ethynyl,

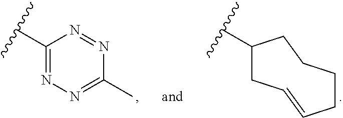

19. (canceled)

20. (canceled)

21. The conjugate of claim 9, wherein R₂ comprise a functional molecule selected from the group consisting of a DNA topoisomerase I inhibitor, a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder,a DHFR inhibitor, a nucleoside analog, a HD AC inhibitor; an anthracycline; a NAMPT inhibitor; a hydrophilic prodrug; a SN-38 glucuronide, a etoposide phosphate; a nitrogen mustard, a proteosome inhibitor, a cytokine, a Toll-like receptor agonist and a Sting agonist.

22. The conjugate of claim 21, wherein R₂ comprises Dxd.

23. The conjugate of claim 9, wherein R₂ comprise a linker which is selected from the group consisting of a cleavable linker, a non-cleavable linker, a hydrophilic linker, a hydrophobic linker, a procharged linker, an uncharged linker and a dicarboxylic acid-based linker.

24. The conjugate of claim 23, wherein R₂ comprise a linker which is selected from the group consisting of VC-PAB, N-succinimidyl-3-(2-pyridyldithio) propionate (SPDP), N-succinimidyl 4-(2-pyridyldithio) pentanoate (SPP), N-succinimidyl 4-(2-pyridyldithio) butanoate (SPDB), N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl)cyclohexanecarboxylate (SMCC), N-sulfosuccinimidyl 4-(maleimidomethyl)cyclohexanecarboxylate (sulfo-SMCC) or 2,5-dioxopyrrolidin-1-yl 17-(2,5-dioxo-2,5-dihydro-1H-pyrrol-1-yl)-5,8,11, 14-tetraoxo-4,7,10,13-tetraazaheptadecan-1-oate (CX1-1).

25. The conjugate of claim 9, wherein a is 2, 4,6 or 8.

26. The conjugate of claim 9, wherein L is selected from the group consisting of: 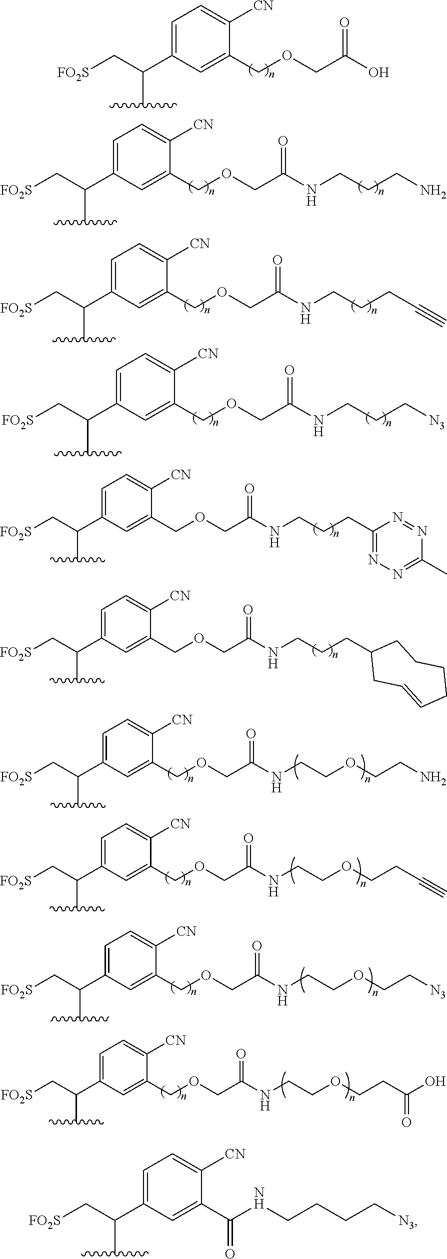
wherein each of n is an integer selected from 1-8.

27. The conjugate of claim 9, wherein L is:

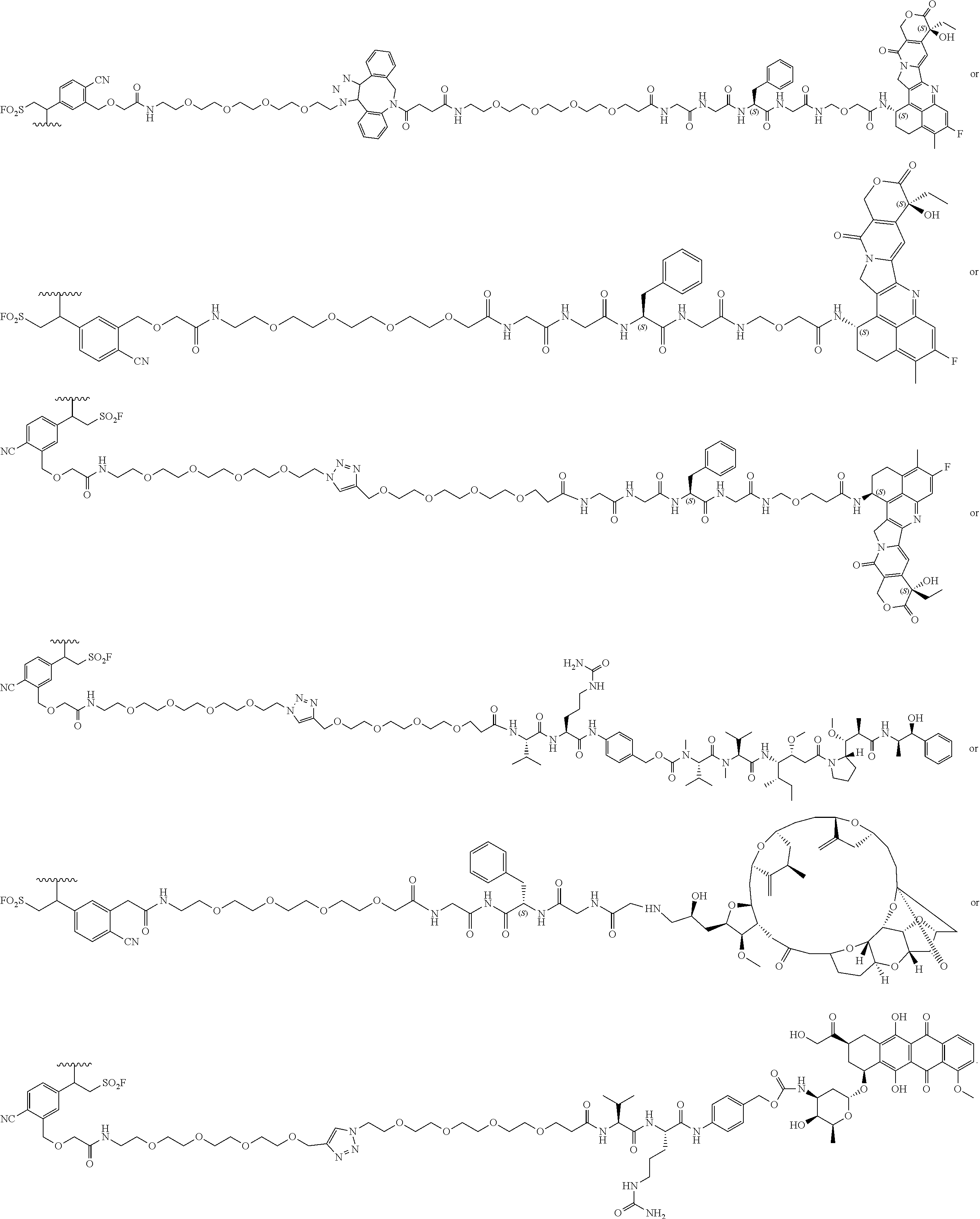

28. (canceled)

29. A method for preparation a conjugate, comprising contacting a compound of formula (Ib): 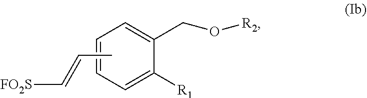
to M, wherein,
M is a protein, which comprising a nucleophilic functional group;
R₁ is an electron withdrawing group selected from —CN, —NO₂, —CHO, fluoro, trifluoro methyl, acetoxy, sulfonic acid, —COR₁₁, and —COOR₁₂, wherein, each of R₁₁ and R₁₂ is independently selected from C₁-C₆ alkyl; and
R₂ comprises a functional molecule selected from azido, alkynyl, cyclohydrocarbyl, alkyne, —NH₂, carboxyl, and tetrazine, or R₂ comprise a functional molecule selected from a toxin, a fluorescent dye, or a biotin.

30. The method of claim 29, wherein the formula (Ib) is

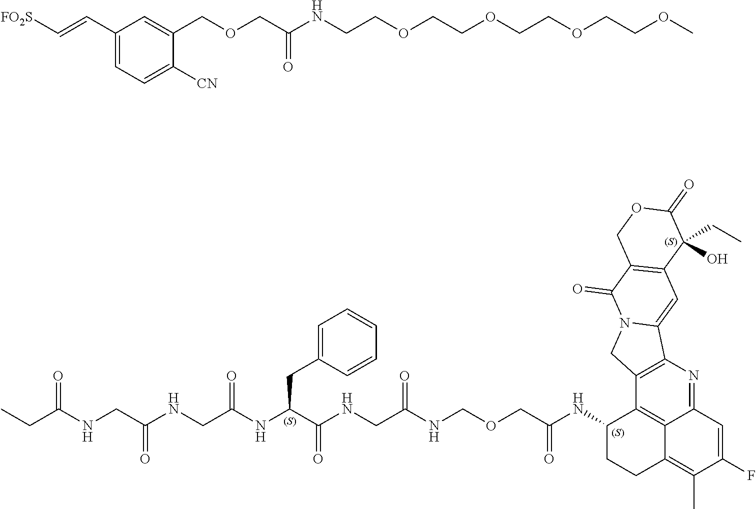

31. (canceled)

32. (canceled)

33. A pharmaceutical composition comprising the conjugate of claim 9 and a pharmaceutically acceptable carrier.

34. (canceled)
